(19) United States
(12) Patent Application Publication
(10) Pub. No.: US 2023/0414786 A1
(43) Pub. Date: Dec. 28, 2023

(54) PERFUSION-GUIDED GENE THERAPY FOR IMPROVING CANCER TREATMENT

(71) Applicants: Board of Regents, The University of Texas System, Austin, TX (US); The University of Chicago, Chicago, IL (US)

(72) Inventors: Shashank R. Sirsi, Frisco, TX (US); Aditi Bellary, Dallas, TX (US); Chance Nowak, Richardson, TX (US); Leonidas Bleris, Allen, TX (US); Sonia L. Hernandez, Chicago, IL (US)

(73) Assignees: Board of Regents, The University of Texas System, Austin, TX (US); The University of Chicago, Chicago, IL (US)

(21) Appl. No.: 18/303,049

(22) Filed: Apr. 19, 2023

Related U.S. Application Data
(60) Provisional application No. 63/332,594, filed on Apr. 19, 2022.

(51) Int. Cl. A61K 48/00; A61K 41/00; A61P 35/00; A61K 45/06

(52) U.S. Cl. CPC A61K 48/0058; A61K 41/0028; A61P 35/00; A61K 48/0091; A61K 45/06

(57) ABSTRACT

The present methods use gene therapy to confer inducible nitric oxide synthase (iNOS) expression solely in the tumor space, using focused ultrasound targeting. NOS catalyzes the reaction that generates nitric oxide (NO), a potent endogenous vasodilator. Microbubble-mediated non-viral delivery overcomes major barriers associated with non-viral NO gene therapy. The methods increase tumor perfusion and compound the efficacy of a vast array of chemotherapy, radiotherapy, and immune-based treatments.

### RELATED APPLICATIONS

This application claims priority to U.S. Provisional Patent Application Ser. No. 63/332,594, filed Apr. 19, 2022, entitled “Perfusion-Guided Gene Therapy for Improving Cancer Treatment,” which is hereby incorporated by reference herein.

### BACKGROUND

This invention was made with government support under Grant No. RO1 CA235756 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Jun. 29, 2023, is named UTD_215595_181SEQID.xml and is 16 kB in size.

This disclosure pertains to the utilization of microbubbles in a focused ultrasound system to improve tumor perfusion in a cancerous tumor space of a subject.

Neuroblastoma (NB) is a pediatric malignancy that accounts for 15% of cancer-related childhood mortality. Neuroblastoma (NB) is the most common extracranial solid tumor afflicting infants and children. High-risk NB requires an aggressive chemoradiotherapy regimen that causes significant off-target toxicity. Current standard-of-care for high-risk disease includes surgical resection, intensive chemoradiotherapy, stem cell transplantation, and immunotherapy. Despite this invasive treatment, many patients either relapse or do not respond adequately. Unfortunately, the estimated 5-year survival rate for patients with high-risk NB is only 50%, and those that achieve cure often manifest chronic adverse effects (including infertility, hearing loss, cardiovascular complications and poor growth) which emanate from off-target toxicities unleashed by the treatment itself. This grim reality highlights the need for targeted approaches to treat this malignancy, ideally increasing the anti-tumor efficacy while limiting systemic toxicity.

Poor prognoses in many cancers are often correlated with high levels of hypoxia and increased interstitial hypertension. Improving perfusion in tumors has the potential to reduce hypoxia and interstitial pressure in solid tumors, leading to improved drug delivery and better therapeutic outcomes. Current approaches to restoring perfusion use anti-angiogenic agents (such as bevacizumab) to ‘normalize’ tumors by curbing unchecked angiogenesis, thereby restoring the aberrant structure and function of the tumor vasculature. However, clinical trials have uncovered major limitations/challenges using anti-angiogenic strategies, most notably that acquired resistance and amplified invasiveness may ensue from blocking the VEGF pathway. Other approaches targeting tumor vasculature to improve tumor perfusion, such as Notch blockade, have led to accelerated metastasis. The prevailing explanation for this cascade of events posits that an incomplete response to anti-angiogenic therapy likely arises from limited bioavailability of therapeutic agent in the tumor mass, meaning that insufficient perfusion ultimately impedes intratumoral deposition.

Nitric oxide (NO) therapy has been proposed as a potential solution to overcome tumor hypoxia and poor perfusion. However NO-based strategies have met with limited success for myriad reasons, and the effects of NO can be unpredictable. For instance, the literature extensively documents NO' s paradoxical and context-dependent activity: at low concentrations, NO boosts carcinogenesis, angiogenesis, and tumor proliferation, while at high concentrations it assists tumor regression by inducing extensive DNA damage, thus shunting cells toward apoptosis. Hence, delivering the precise concentration of NO to elicit a therapeutic response is critical. More importantly, NO is readily scavenged by hemoglobin in erythrocytes; in fact, it has a much greater affinity for hemoglobin than does oxygen and its half-life in whole blood is a mere 1.8 ms.

Given its short half-life and role as a ubiquitous signaling molecule, elevating systemic NO levels is neither practical nor desirable. Thus, strategies to exploit NO as a cancer therapy require localized delivery or production within the tumor space to restrict its bioeffects to this region. Several recent studies have developed NO carriers or “donor” molecules to improve the circulation half-life and efficacy of NO in cancer therapy, such as organic nitrates, nitrosometal complexes, N-diazeniumdiolates, furoxans, nitrosothiols, RRX-001, and L-arginine. Polymeric nanoparticle-based NO donors have also been shown to enhance tumor perfusion and to increase permeability and retention in cancer therapy. However, the lack of targeting capabilities and off-target accumulation of such donors may curtail their viability in vivo.

### SUMMARY

The present disclosure relates generally to a novel targeted non-viral gene therapy strategy to enhance tumor perfusion.

In particular, the present disclosure relates to the use of gene therapy to confer inducible nitric oxide synthase (iNOS) expression solely in the tumor space, using focused ultrasound targeting. NOS catalyzes the reaction that generates nitric oxide (NO), a potent endogenous vasodilator. A targeted non-viral image-guided platform delivers iNOS-expressing plasmid DNA (pDNA) to vascular endothelial cells encasing tumor blood vessels. Following transfection, longitudinal quantitative contrast-enhanced ultrasound (qCEUS) imaging reveals an increase in tumor perfusion over 72 hours, attributed to elevated intratumoral iNOS expression. Transiently increasing tumor perfusion improves liposome-encapsulated chemotherapeutic uptake and distribution. The iNOS gene delivery paradigm described herein can also significantly improve radio and immunotherapies by increasing the delivery of liposome-encapsulated radiosensitizers and immunomodulators, potentially improving upon current neuroblastoma treatment without concomitant adverse effects. qCEUS imaging can effectively monitor changes in tumor perfusion in vivo, allowing the identification of an ideal time-point to administer therapy.

### BRIEF DESCRIPTION OF THE DRAWINGS

FIG. 1A shows a schematic of preferred embodiments of the procedures and mechanisms of action of iNOS gene therapy as described herein.

FIG. 1B shows a plasmid map for an iNOS integration vector according to preferred

embodiments disclosed herein.

FIG. 2A shows sequence maps of mKate (“Sham”) and iNOS plasmids, in accordance with preferred embodiments described herein, FIG. 2B shows decreased cell proliferation in NGP cells transfected with iNOS followed by increasing L-DOX concentrations compared to Sham transfected cells, and FIG. 2C shows increase in protein extracts in iNOS transfected cells compared to Sham transfected cells.

FIG. 3A shows confocal microscopy images revealing elevated iNOS expression in tumor endothelium after iNOS transfection compared to untreated or Sham transfected tumors, FIG. 3B shows representative images of glomeruli (labeled “G”) adjacent to NGP tumors, FIG. 3C shows quantification of the iNOS signal intensity showing a greater than two-fold increase within the lectin-positive area in iNOS transfected tumors compared to untreated or Sham transfected tumors, and FIG. 3D shows iNOS expression in adjacent glomeruli remains unchanged after iNOS or Sham transfections.

FIG. 4A shows a schematic representation of the workflow for generating 3D tumor reconstructions from 2D contrast images, FIG. 4B shows representative 3D contrast images displaying the increase in perfusion-volume post-treatment in comparison with Sham-treated and untreated controls, FIG. 4C shows normalized tumor perfusion volumes are plotted over a 7-day period, demonstrating a significant increase in perfusion post-treatment, FIG. 4D shows time-intensity curves derived from non-linear 2D imaging to assess perfusion dynamics in NB tumors, FIG. 4E shows flash-destruction (FD) replenishment curves reveal augmented perfusion rates in iNOS-treated tumors compared to controls, and FIG. 4F shows quantitative analysis of normalized tumor 2D re-perfusion rates after FD indicating enhanced perfusion in response to iNOS treatment over the 7-day time course.

FIG. 5A shows images of tumors, indicating that iNOS transfection reduced tumor hypoxia as indicated by a decrease in pimonidazole immunoreactivity relative to Sham-transfected tumors, and FIG. 5B shows quantification of pimonidazole and lectin stains (left) demonstrating decreased hypoxia in iNOS tumors.

FIG. 6A shows a summary of the methodology for priming tumors with iNOS before treatment, FIG. 6B shows that pretreatment of tumors with iNOS significantly increased drug uptake, as demonstrated by a 3.3-fold increase in doxorubicin accumulation compared to untransfected counterparts, and 2.3-fold boost compared to tumors receiving Sham treatment, FIG. 6C shows representative images of doxorubicin uptake revealing that Sham tumors had slightly higher uptake compared to controls, while iNOS tumors had significantly higher uptake than both groups, and FIG. 6D shows that quantification of TUNEL areas normalized by total tumor area showed that iNOS-transfected tumors had significantly higher levels of apoptosis compared to untreated controls receiving L-DOX without sonopermeation or untransfected tumors given L-DOX with sonopermeation.

FIG. 7A shows a summary of the procedure adopted to prime tumors with gene therapy before low dosage chemotherapy plus sonopermeation, FIG. 7B shows that iNOS transfection significantly increases survival time in NGP tumors, and FIG. 7C shows the change in percentage body weight for each treatment group.

### DETAILED DESCRIPTION OF PREFERRED EMBODIMENTS

The present disclosure relates to non-viral delivery of inducible nitric oxide synthase (iNOS) to tumor vascular endothelial cells using ultrasound guidance. Localized iNOS gene therapy favorably alters the vascular properties of neuroblastoma (NB) to improve tumor sensitivity to sonopermeation with liposomal nanodrugs.

Preferred embodiments described herein relate to the use of gene therapy to confer inducible nitric oxide synthase (iNOS) expression solely in the tumor space, using focused ultrasound targeting. NOS catalyzes the reaction that generates nitric oxide (NO), a potent endogenous vasodilator. NOS are a class of enzymes that produce NO from oxidation of the substrate L-arg (L-arginine) in a nicotinamide adenine dinucleotide phosphate (NADPH)-dependent reaction. The iNOS variant of nitric oxide synthase is the most potent form and not naturally present in most vascular endothelial cells. Therapeutic benefits of iNOS expression in tumors using exogenous genes have previously been explored in vitro and in vivo. The in vivo experiments highlighted the potential of sensitizing tumors through iNOS and gene transfection, but to prevent off-target effects they were achieved by infusing plasmid DNA (pDNA) directly into the tumor. iNOS gene therapy has not been explored in treating high-risk NB or other pediatric solid tumors.

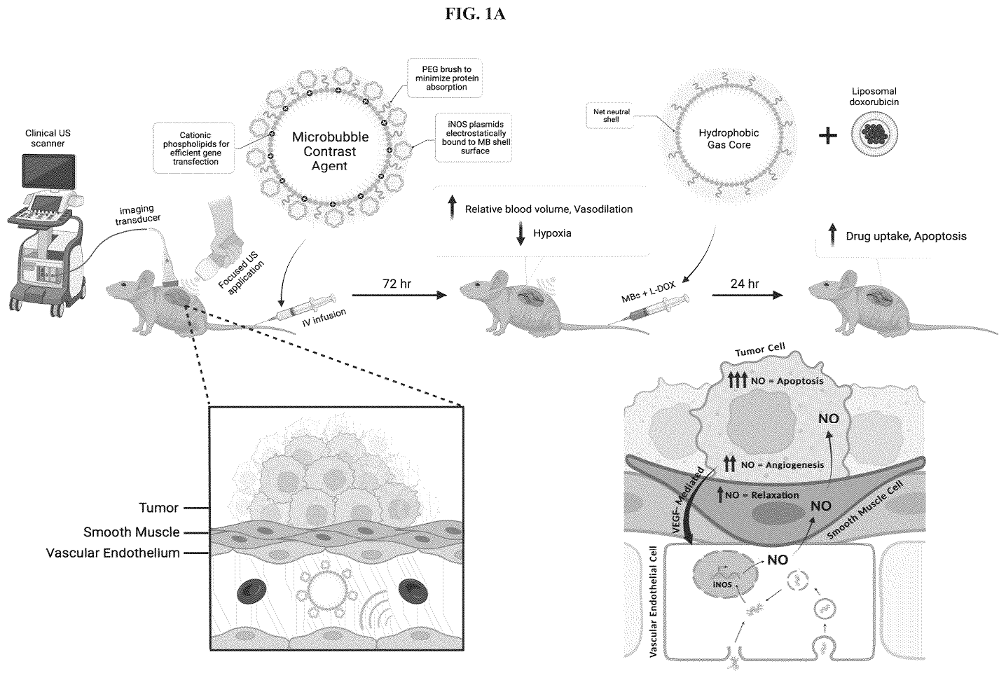

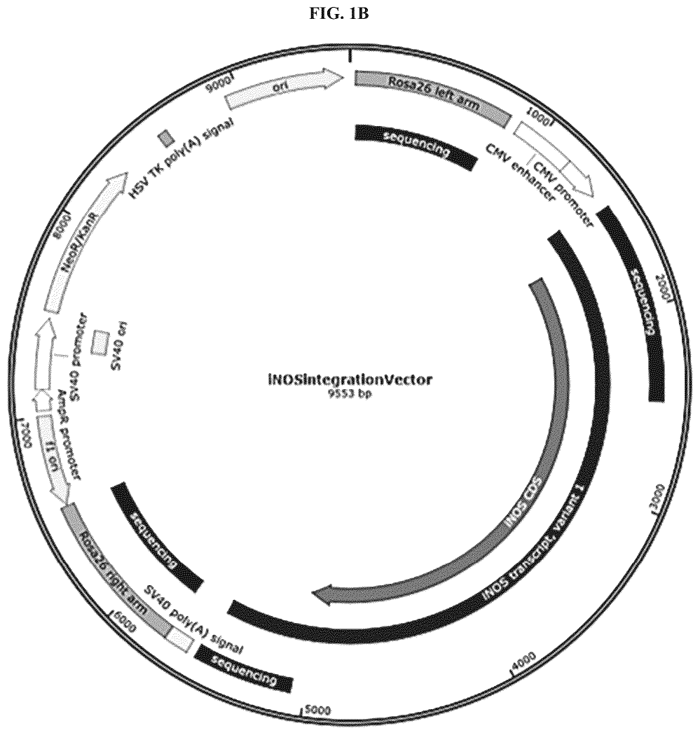

FIG. 1A shows a schematic of preferred embodiments of the procedures and mechanisms of action of iNOS gene therapy as described herein.

A targeted non-viral image-guided platform is described herein for delivery of iNOS-expres sing plasmid DNA (pDNA) to vascular endothelial cells encasing tumor blood vessels. To construct a gene delivery vector, in preferred embodiments, cationic ultrasound responsive agents (known as “microbubbles”) are preferably employed to carry pDNA in circulation and transfect tumor vascular endothelial cells in vivo using focused ultrasound (FUS) energy. Microbubbles (MBs) are gaseous spheres enclosed within a phospholipid shell, the presence of which attenuates gas diffusion out of the bubble. They typically span 1 to 10 μm, making them smaller than the ultrasonic wavelengths used in medical imaging, and as such they serve as point scatterers rather than reflectors of ultrasound. Due to the compressibility of their gas core, MBs volumetrically expand and contract in phase with external pressure changes caused by a sound wave. At high acoustic pressures, inertial forces triggered by MB implosion can rupture cell membranes (reversibly) and provide direct access to the endothelial cell cytoplasm. Therefore, by judiciously applying focused ultrasound (FUS), MBs' interaction with ultrasound can be spatiotemporally fine-tuned to achieve site-specific release of plasmids in vivo, a technique referred to as “sonoporation” or “sonopermeation.” By combining FUS therapy with cationic ultrasound contrast agents (UCAs), selective intratumoral transfection of pDNA encoding the iNOS enzyme is achieved.

Localized iNOS gene therapy can favorably alter the vascular properties of neuroblastoma to improve tumor sensitivity to sonopermeation with liposomal nanodrugs. Following transfection, longitudinal quantitative contrast-enhanced ultrasound (qCEUS) imaging revealed an increase in tumor perfusion over 72 hours, attributed to elevated intratumoral iNOS expression. While transitory, the degree of expression was sufficient to induce significant increases in tumoral perfusion and to appreciably enhance chemotherapeutic payload and extend survival time in an orthotopic xenograft model. Long-term quantitative contrast enhanced US (qCEUS) is preferably used to monitor changes in tumor perfusion in vivo, allowing identification of an ideal time-point to administer therapy, such as administration of liposomal doxorubicin (L-DOX) chemotherapy to increase its delivery and retention. The microbubble' s dual functionality allows it to serve as a theranostic tool to gauge increases in tumor perfusion stemming from raised intratumoral NO levels.

Preferred embodiments of the methods described herein represent clinically viable solutions that improve neuroblastoma response to therapy without increasing side effects. The techniques (1) improve non-viral gene delivery to tumors, (2) monitor dynamic changes in the tumor vasculature in response to NO treatment, and (3) bestow control of tumor vascular properties in vivo while enabling real-time feedback to determine when tumors are primed for primary treatment. Microbubble-mediated non-viral transfection of vascular endothelial cells as described herein is an effective approach to enhancing tumor perfusion and liposomal drug accumulation and could have a significant impact on other pediatric and adult solid tumors.

The present methods overcome poor tumor perfusion and compound the efficacy of a vast array of chemotherapy, radiotherapy, and immune-based treatments. Despite the emphasis on neuroblastoma, the present methods represent a versatile technology that is clinically translatable to a wide range of pediatric and adult solid tumors. iNOS gene therapy was used to channel high levels of NO that can be produced per enzyme. Moreover, microbubble-mediated non-viral delivery is a rational approach to overcome major barriers associated with non-viral NO gene therapy.

### EXAMPLES

### Materials and Methods

Preparation of Microbubbles. Net neutral MBs for imaging tumor perfusion were formulated using a lipid film composed of 14.34 mg of 1,2-distearoyl-sn-glycero-3-phosphocholine (DSPC, 790.16 MW) and 5.66 mg of N-(methylpolyoxyethylene oxycarbonyl)-1,2-distearoyl-sn-glycero-3-phosphoethanolamine (DSPE-PEG2000, 2805.97 MW) (NOF Corporation, Tokyo, Japan), dissolved in chloroform (Sigma-Aldrich, St. Louis, Mo.). Cationic MBs for electrostatically binding pDNA were likewise fabricated using a lipid film comprising 11.35 mg of DSPC, 5.76 mg of DSPE-PEG2K, and 2.88 mg of 1,2-stearoyl-3-trimethylammonium-propane (DOTAP, 702.57 MW) (Avanti Polar Lipids, Alabaster, Ala.). The lipid solution was evaporated for 48 h and then stored as a lipid film in a sealed scintillation vial at −20° C. On the day of intended use, the 20 mg film was diluted to 2 mg/mL (10 mL total) in a filtered mixture of 10% propane-1,2-diol (propylene glycol, 76.1 FW) (v/v), 10% propane-1,2,3-triol (glycerol, 92.09 FW) (v/v), and 10× phosphate buffer saline (PBS) diluted to lx (Fisher Scientific, Waltham, Mass.). The lipid solution was heated to 65° C. on an Isotemp Heating Block and bath sonicated in a 1.9 L Ultrasonic Bath Sonicator (Fisher Scientific, Waltham, Mass.) until the lipid was completely suspended. High-concentration MBs were generated using a probe micro-tip sonication method previously described by Feshitan et al. The heated lipid solution was placed in contact with the sonicator tip (Branson 450 Ultrasonics Sonifier with microtip attachment, Emerson, St. Louis, Mo.) and operated at 70% power under constant flushing with Decafluorobutane (PFB, 238 MW, FluoroMed LP, Round Rock, Tex.) for 10 s. The combined lipid suspension was supercooled in an ice bath and then washed three times in a 10 mL Luer tip syringe (BD, Franklin Lakes, N.J.) at 300 relative centrifugal force (RCF) for 3 min in a Bucket Centrifuge Model 5804R (Eppendorf, Hauppauge, N.Y.) to collect the bubbles. The MBs were characterized using a Multisizer 4e Coulter Counter (MS4, Beckman Coulter, Brea, Calif.) to ensure the median bubble size was 1.90±0.925 μm. The pDNA adsorption properties of the cationic microbubbles used in this study have been previously established in the literature, and estimated to be 0.05 pg/μm².

Preparation of Plasmid DNA. The mKate expression vector was from a modified pmKate2-C vector (Evrogen #FP181) that was custom designed for integration into the rosa26 genomic safe harbor locus. The iNOS expression vector was constructed by replacing the mKate2 ORF with M. musculus-derived pBS-iNOS, which was a gift from Charles Lowenstein (Addgene plasmid #19295). The transcript was cloned using PCR with Q5 ® High-Fidelity DNA Polymerase (NEB, #M0492S) and primers P1: cagtagaccggtgagactctggccccacgggacacag (SEQ ID NO: 1) and P2: cagtagcaattggaattgtaatacgactcactatagg (SEQ ID NO:2). The PCR amplicon containing the iNOS transcript and the mKate2 expression vector was digested with Agel-HF (NEB, #R3552S) and Mfel-HF (NEB, #R3589S). The iNOS transcript was cloned into the expression vector with T4 DNA Ligase (NEB, #M0202S), replacing the mKate2 ORF. Although genetic elements for homology recombination are present in both plasmids, integration was not assessed in this study, and all results are believed to originate from the transient plasmid expression. The full sequence for the iNOS plasmid used in this example is as follows:

(SEQ ID NO: 3)
ggccttttgctggccttttgctcacatgtcagttaaCGGCAGCCGGAGTGCGCAGCCGCCGGCAGCC
TCGCTCTGCCCACTGGGTGGGGGGGGAGGTAGGTGGGGTGAGGCGAGCTGGACGTGCGGGCGCGGTC
GGCCTCTGGCGGGGCGGGGGAGGGGAGGGAGGGTCAGCGAAAGTAGCTCGCGCGCGAGCGGCCGCCC
ACCCTCCCCTTCCTCTGGGGGAGTCGTTTTACCCGCCGCCGGCCGGGCCTCGTCGTCTGATTGGCTC
TCGGGGCCCAGAAAACTGGCCCTTGCCATTGGCTCGTGTTCGTGCAAGTTGAGTCCATCCGCCGGCC
AGCGGGGGCGGCGAGGAGGCGCTCCCAGGTTCCGGCCCTCCCCTCGGCTCCGCGCCGCAGAGTCTGG
CCGCGCGCCCCTGCGCAACGTGGCAGGAAGCGCGCGCTGGGGGCGGGGACGGGCAGTAGGGCTGAGC
GGCTGCGGGGGGGGTGCAAGCACGTTTCCGACTTGAGTTGCCTCAAGAGGGGCGTGCTGAGCCAGAC
CTCCATCGCGCACTCCGGGGAGTGGAGGGAAGGAGCGAGGGCTCAGTTGGGCTGTTTTGGAGGCAGG
AAGCACTTGCTCTCCCAAAGTCGCTCTGAGTTGTTATCAGTAAGGGAGCTGCAGTGGAGTAGGCGGG
GAGAAGGCCGCACCCTTCTCCGGAGGGGGGAGGGGAGTGTTGCAATACCTTTCTGGGAGTTCTCTGC
TGCCTCCTGGCTTCTGAGGACCGCCCTGGGCCTGGGAGAATCCCTtccccctcttccctcgtgatct
gcaactccagtctttctagattaatagtaatcaattacggggtcattagttcatagcccatatatgg
agttccgcgttacataacttacggtaaatggcccgcctggctgaccgcccaacgacccccgcccatt
gacgtcaataatgacgtatgttcccatagtaacgccaatagggactttccattgacgtcaatgggtg
gagtatttacggtaaactgcccacttggcagtacatcaagtgtatcatatgccaagtacgcccccta
ttgacgtcaatgacggtaaatggcccgcctggcattatgcccagtacatgaccttatgggactttcc
tacttggcagtacatctacgtattagtcatcgctattaccatggtgatgcggttttggcagtacatc
aatgggcgtggatagcggtttgactcacggggatttccaagtctccaccccattgacgtcaatggga
gtttgttttggcaccaaaatcaacgggactttccaaaatgtcgtaacaactccgccccattgacgca
aatgggcggtaggcgtgtacggtgggaggtctatataagcagagctggtttagtgaaccgtcagatc
cgctagcgctaccggtcgagactctggccccacgggacacagtgtcactggtttgaaaCTTCTCAGC
CACCTTGGTGAAGGGACTGAGCTGTTAGAGACACTTCTGAGGCTCCTCACGCTTGGGTCTTGTTCAC
TCCACGGAGTAGCCTAGTCAACTGCAAGAGAACGGAGAACGTTGGATTTGGAGCAGAAGTGCAAAGT
CTCAGACATGGCTTGCCCCTGGAAGTTTCTCTTCAAAGTCAAATCCTACCAAAGTGACCTGAAAGAG
GAAAAGGACATTAACAACAACGTGAAGAAAACCCCTTGTGCTGTTCTCAGCCCAACAATACAAGATG
ACCCTAAGAGTCACCAAAATGGCTCCCCGCAGCTCCTCACTGGGACAGCACAGAATGTTCCAGAATC
CCTGGACAAGCTGCATGTGACATCGACCCGTCCACAGTATGTGAGGATCAAAAACTGGGGCAGTGGA
GAGATTTTGCATGACACTCTTCACCACAAGGCCACATCGGATTTCACTTGCAAGTCCAAGTCTTGCT
TGGGGTCCATCATGAACCCCAAGAGTTTGACCAGAGGACCCAGAGACAAGCCTACCCCTCTGGAGGA
GCTCCTGCCTCATGCCATTGAGTTCATCAACCAGTATTATGGCTCCTTTAAAGAGGCAAAAATAGAG
GAACATCTGGCCAGGCTGGAAGCTGTAACAAAGGAAATAGAAACAACAGGAACCTACCAGCTCACTC
TGGATGAGCTCATCTTTGCCACCAAGATGGCCTGGAGGAATGCCCCTCGCTGCATCGGCAGGATCCA
GTGGTCCAACCTGCAGGTCTTTGACGCTCGGAACTGTAGCACAGCACAGGAAATGTTTCAGCACATC
TGCAGACACATACTTTATGCCACCAACAATGGCAACATCAGGTCGGCCATCACTGTGTTCCCCCAGC
GGAGTGACGGCAAACATGACTTCAGGCTCTGGAATTCACAGCTCATCCGGTACGCTGGCTACCAGAT
GCCCGATGGCACCATCAGAGGGGATGCTGCCACCTTGGAGTTCACCCAGTTGTGCATCGACCTaggc
tggaagccccgctatggccgctttgatgtgctgcctctggtcttgcaagctgatggtcaagatccag
aggtctttgaaatccctcctgatcttgtgttggaggtgaccatggagcatcccaagtacgagtggtt
ccaggagctcgggttgaagtggtatgcactgcctgccgtggccaacatgctactggaggtgggtggc
ctcgaattcccagcctgccccttcaatggttggtacatgggcaccgagattggagttcgagacttct
gtgacacacagcgctacaacatcctggaggaagtgggccgaaggatgggcctggagacccacacact
ggcctccctctggaaagaccgggctgtcacggagatcaatgtggctgtgctccatagtttccagaag
cagaatgtgaccatcatggaccaccacacagcctcagagtccttcatgaagcacatgcagaatgagt
accgggcccgtggaggctgcccggcagactggatttggctggtccctccagtgtctgggagcatcac
ccctgtgttccaccaggagatgttgaactatgtcctatctccattctactactaccagatcgagccc
tggaagacccacatctggcagaatgagaagctgaggcccaggaggagagagatccgatttagagtct
tggtgaaagtggtgttctttgcttccatgctaatgcgaaaggtcatggcttcacgggtcagagccac
agtcctctttgctactgagacagggaagtctgaagcactagccagggacctggccaccttgttcagc
tacgccttcaacaccaaggttgtctgcatggaccagtataaggcaagcaccttggaagaggagcaac
tactgctggtggtgacaagcacatttgggaatggagactgtcccagcaatgggcagactctgaagaa
atctctgttcatgcttagagaactcaaccacaccttcaggtatgctgtgtttggccttggctccagc
atgtaccctcagttctgcgcctttgctcatgacatcgaccagaagctgtcccacctgggagcctctc
agcttgccccaacaggagaaggggacgaactcagtgggcaggaggatgccttccgcagctgggctgt
acaaaccttccgggcagcctgtgagacctttgatgtccgaagcaaacatcacattcagatcccgaaa
cgcttcacttccaatgcaacatgggagccacagcaatataggctcatccagagcccggagcctttag
acctcaacagagccctcagcagcatccatgcaaagaacgtgtttaccatgaggctgaaatcccagca
gaatctgcagagtgaaaagtccagccgcaccaccctcctcgttcagctcaccttcgagggcagccga
gggcccagctacctgcctggggaacacctggggatcttcccaggcaaccagaccgccctggtgcagg
gaatcttggagcgagttgtggattgtcctacaccacaccaaactgtgtgcctggaggttctggatga
gagcggcagctactgggtcaaagacaagaggctgcccccctgctcactcagccaagccctcacctac
ttcctggacattacgacccctcccacccagctgcagctccacaagctggctcgctttgccacggacg
agacggataggcagagattggaggccttgtgtcagccctcagagtacaatgactggaagttcagcaa
caaccccacgttcctggaggtgcttgaagagttcccttccttgcatgtgcccgctgccttcctgctg
tcgcagctccctatcttgaagccccgctactactccatcagctectcccaggaccacaccccctcgg
aggttcacctcactgtggccgtggtcacctaccgcacccgagatggtcagggtcccctgcaccatgg
tgtctgcagcacttggatcaggaacctgaagccccaggacccagtgccctgctttgtgcgaagtgtc
agtggcttccagctccctgaggacccctcccagccttgcatcctcattgggcctggtacgggcattg
ctcccttccgaagtttctggcagcageggctccatgactcccagcacaaagggctcaaaggaggccg
catgagcttggtgtttgggtgccggcacccggaggaggaccacctctatcaggaagaaatgcaggag
atggtccgcaagagagtgctgttccaggtgcacacaggctactcccggctgcccggcaaacccaagg
tctacgttcaggacatcctgcaaaagcagctggccaatgaggtactcagcgtgctccacggggagca
gggccacctctacatttgcggagatgtgcgcatggctcgggatgtggctaccacattgaagaagctg
gtggccaccaagctgaacttgagcgaggagcaggtggaagactatttcttccagctcaagagccaga
aacgttatcatgaagatatcttcggtgcagtcttttcctatggggcaaaaaagggcagcgccttgga
ggagcccaaagccacgaggctctgacagcccagagttccagCTTCTGGCACTGAGTAAAGATAATGG
TGAGGGGCTTGGGGAGACAGCGAAATGCAATCCCCCCCAAGCCCCTCATGTCATTCCCCCCTCCTCC
ACCCTACCAAGTAGTATTGTACTATTGTGGACTACTAAATCTCTCTCCTCTCCTCCCTCCCCTCTCT
CCCTTTCCTCCCTTCTTCTCCACTCCCCAGCTCCCTCCTTCTCCTTCTCCTCCTTTGCCTCTCACTC
TTCCTTGGAGCTGAGAGCAGAGAAAAACTCAACCTCCTGACTGAAGCACTTTGGGTGACCACCAGGA
GGCACCATGCCGCCGCTCTAATACTTAGCTGCACTATGTACAGATATTTATACTTCATATTTAAGAA
AACAGATACTTTTGTCTACTCCCAATGATGGCTTGGGCCTTTCCTGTATAATTCCTTGATGAAAAAT
ATTTATATAAAATACATTTTATTTTAATCAAAAAAAAAAAAGCGGCCGCCACCGCGGTGGAGCTCCA
ATtcgccctatagtgagtcgtattacaattccaattgttgttgttaacttgtttattgcagcttata
atggttacaaataaagcaatagcatcacaaatttcacaaataaagcatttttttcactgcattctag
ttgtggtttgtccaaactcatcaatgtatcttaacgcgtgatgggggGAGTCTTCTGGGCAGGCTTA
AAGGCTAACCTGGTGTGTGGGCGTTGTCCTGCAGGGGAATTGAACAGGTGTAAAATTGGAGGGACAA
GACTTCCCACAGATTTTCGGTTTTGTCGGGAAGTTTTTTAATAGGGGCAAATAAGGAAAATGGGAGG
ATAGGTAGTCATCTGGGGTTTTATGCAGCAAAACTACAGGTTATTATTGCTTGTGATCCGCCTCGGA
GTATTTTCCATCGAGGTAGATTAAAGACATGCTCACCCGAGTTTTATACTCTCCTGCTTGAGATCCT
TACTACAGTATGAAATTACAGTGTCGCGAGTTAGACTATGTAAGCAGAATTTTAATCATTTTTAAAG
AGCCCAGTACTTCATATCCATTTCTCCCGCTCCTTCTGCAGCCTTATCAAAAGGTATTTTAGAACAC
TCATTTTAGCCCCATTTTCATTTATTATACTGGCTTATCCAACCCCTAGACAGAGCATTGGCATTTT
CCCTTTCCTGATCTTAGAAGTCTGATGACTCATGAAACCAGACAGATTAGTTACATACACCACAAAT
CGAGGCTGTAGCTGGGGCCTCAACACTGCAGTTCTTTTATAACTCCTTAGTACACTTTTTGTTGATC
CTTTGCCTTGATCCTTAATTTTCAGTGTCTATCACCTCTCCCGTCAGGTGGTGTTCCACATTTGGGC
CTATTCTCAGTCCAGGGAGTTTTACAACAATAGATGTATTGAGAATCCAACCTAAAGCTTAACTTTC
CACTCCCATGAATGCCTCTCTCCTTTTTCTCCATTTATaaactgacgcgtaaattgtaagcgttaat
attttgttaaaattcgcgttaaatttttgttaaatcagctcattttttaaccaataggccgaaatcg
gcaaaatcccttataaatcaaaagaatagaccgagatagggttgagtgttgttccagtttggaacaa
gagtccactattaaagaacgtggactccaacgtcaaagggcgaaaaaccgtctatcagggcgatggc
ccactacgtgaaccatcaccctaatcaagttttttggggtcgaggtgccgtaaagcactaaatcgga
accctaaagggagcccccgatttagagcttgacggggaaagccggcgaacgtggcgagaaaggaagg
gaagaaagcgaaaggagcgggcgctagggcgctggcaagtgtagcggtcacgctgcgcgtaaccacc
acacccgccgcgcttaatgcgccgctacagggcgcgtcaggtggcacttttcggggaaatgtgcgcg
gaacccctatttgtttatttttctaaatacattcaaatatgtatccgctcatgagacaataaccctg
ataaatgcttcaataatattgaaaaaggaagagtcctgaggcggaaagaaccagctgtggaatgtgt
gtcagttagggtgtggaaagtccccaggctccccagcaggcagaagtatgcaaagcatgcatctcaa
ttagtcagcaaccaggtgtggaaagtccccaggctccccagcaggcagaagtatgcaaagcatgcat
ctcaattagtcagcaaccatagtcccgcccctaactccgcccatcccgcccctaactccgcccagtt
ccgcccattctccgccccatggctgactaattttttttatttatgcagaggccgaggccgcctcggc
ctctgagctattccagaagtagtgaggaggcttttttggaggcctaggcttttgcaaagatcgatca
agagacaggatgaggatcgtttcgcatgattgaacaagatggattgcacgcaggttctccggccgct
tgggtggagaggctattcggctatgactgggcacaacagacaatcggctgctctgatgccgccgtgt
tccggctgtcagcgcaggggcgcccggttctttttgtcaagaccgacctgtccggtgccctgaatga
actgcaagacgaggcagcgcggctatcgtggctggccacgacgggcgttccttgcgcagctgtgctc
gacgttgtcactgaagcgggaagggactggctgctattgggcgaagtgccggggcaggatctcctgt
catctcaccttgctcctgccgagaaagtatccatcatggctgatgcaatgcggcggctgcatacgct
tgatccggctacctgcccattcgaccaccaagcgaaacatcgcatcgagcgagcacgtactcggatg
gaagccggtcttgtcgatcaggatgatctggacgaagagcatcaggggctcgcgccagccgaactgt
tcgccaggctcaaggcgagcatgcccgacggcgaggatctcgtcgtgacccatggcgatgcctgctt
gccgaatatcatggtggaaaatggccgcttttctggattcatcgactgtggccggctgggtgtggcg
gaccgctatcaggacatagcgttggctacccgtgatattgctgaagagcttggcggcgaatgggctg
accgcttcctcgtgctttacggtatcgccgctcccgattcgcagcgcatcgccttctatcgccttct
tgacgagttcttctgagcgggactctggggttcgaaatgaccgaccaagcgacgcccaacctgccat
cacgagatttcgattccaccgccgccttctatgaaaggttgggcttcggaatcgttttccgggacgc
cggctggatgatcctccagcgcggggatctcatgctggagttcttcgcccaccctagggggaggcta
actgaaacacggaaggagacaataccggaaggaacccgcgctatgacggcaataaaaagacagaata
aaacgcacggtgttgggtcgtttgttcataaacgcggggttcggtcccagggctggcactctgtcga
taccccaccgagaccccattggggccaatacgcccgcgtttcttccttttccccaccccacccccca
agttcgggtgaaggcccagggctcgcagccaacgtcggggcggcaggccctgccatagcctcaggtt
actcatatatactttagattgatttaaaacttcatttttaatttaaaaggatctaggtgaagatcct
ttttgataatctcatgaccaaaatcccttaacgtgagttttcgttccactgagcgtcagaccccgta
gaaaagatcaaaggatcttcttgagatcctttttttctgcgcgtaatctgctgcttgcaaacaaaaa
aaccaccgctaccagcggtggtttgtttgccggatcaagagctaccaactctttttccgaaggtaac
tggcttcagcagagcgcagataccaaatactgtccttctagtgtagccgtagttaggccaccacttc
aagaactctgtagcaccgcctacatacctcgctctgctaatcctgttaccagtggctgctgccagtg
gcgataagtcgtgtcttaccgggttggactcaagacgatagttaccggataaggcgcagcggtcggg
ctgaacggggggttcgtgcacacagcccagcttggagcgaacgacctacaccgaactgagataccta
cagcgtgagctatgagaaagcgccacgcttcccgaagggagaaaggcggacaggtatccggtaagcg
gcagggtcggaacaggagagcgcacgagggagcttccagggggaaacgcctggtatctttatagtcc
tgtcgggtttcgccacctctgacttgagcgtcgatttttgtgatgctcgtcaggggggcggagccta
tggaaaaacgccagcaacgcggcctttttacggttcct

FIG. 1B shows a plasmid map for the exemplary iNOS integration vector used as described herein, according to preferred embodiments.

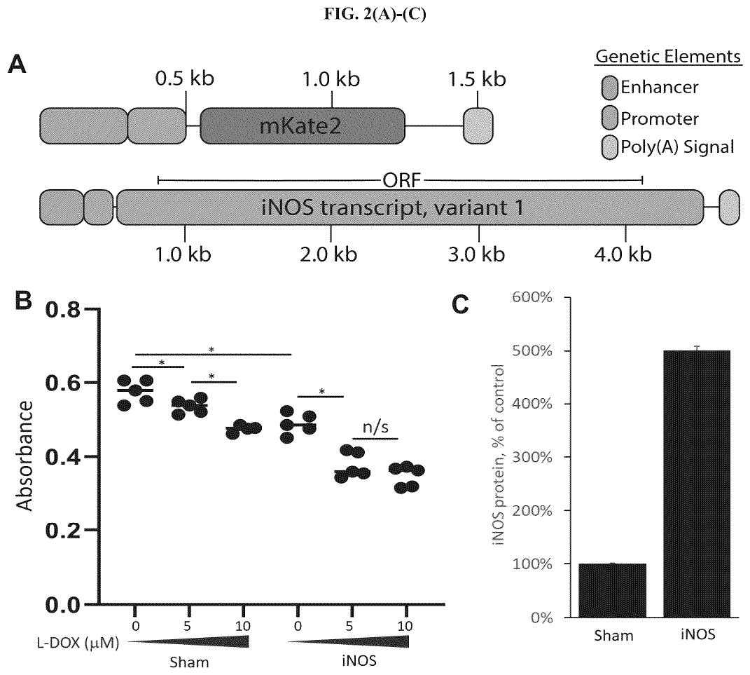

FIG. 2 relates to iNOS and Sham plasmids interacting with L-DOX in NGP tumor cells. FIG. 2A shows sequence maps of mKate and iNOS plasmids, which were deposited to the NGP tumoral vascular endothelium via MB-mediated transfection. Negatively charged pDNA electrostatically binds to cationic bubbles when commingled; this complexation shields the genetic material from being degraded in circulation. Enhancer and promoter elements are derived from CMV, and the poly(A) signals are derived from SV40 virus. FIG. 2B shows in vitro NGP cells transfected with Sham or iNOS followed by increasing L-DOX concentrations demonstrate that iNOS decreases cell proliferation compared to Sham in the absence of L-DOX (red bar), and that low dose L-DOX synergizes with iNOS to further decrease NGP cell proliferation (n=5 per group). FIG. 2C shows that protein extracts interrogated with ELISA harvested from parallel experiments indicate that iNOS transfection leads to a 5-fold iNOS upregulation compared to Sham (n=3 per group).

Orthotopic NGP Tumor Model and Implantation. NGP cells were MYC-N amplified, and thus function as an appropriate model for poor prognosis NB. They reproduce many features of clinical NB, such as histology, frequency, and location of metastatic lesions when renally implanted, as was done in nude athymic mice (Charles River, Wilmington, Mass.) to generate tumor models for this study. Mice were firstly anesthetized with inhalable isoflurane. After being positioned in a sterile environment, the entire right side of each mouse was cleaned with ethanol and painted with Betadine. A 3-5 mm diagonal incision was made with a scalpel blade toward the ribcage atop the kidney. The underlying fascia was cut with scissors to expose the right kidney. A 27-gauge needle (of length 1.3 cm, BD Biosciences) fitted to a syringe containing 20 μL of cell suspension (1×10⁶ NGP cells in Phosphate Buffered Saline, Leibniz Institute DSMZ-GmbH, Braunschweig, Germany) was inserted into the kidney and its contents injected slowly. The kidney was then returned to the abdominal cavity. The fascia was closed with absorbable sutures, followed by staples to seal the skin. Mice were monitored daily to confirm complete recovery, and tumors were allowed to grow for 4-5 weeks (1-2-g weight) before ultrasound experiments were initiated.

Mouse Preparation for Imaging and Sonopermeation. All procedures were performed in accordance with the guidelines stipulated in a protocol approved by the Institutional Animal Care and Use Committee (IACUC) at the University of Texas at Dallas. Mice were anesthetized with 1-2% isoflurane (Vedco, St. Joseph, Mo.) and restrained in the prone position. After confirming the depth of anesthesia by toe pinch, the animals were catheterized via either the left or right lateral tail vein using a winged butterfly infusion set (Terumo Corporation, Tokyo, Japan). Whole body temperature of the mice was maintained at 37° C. using a closed loop temperature control system comprising a heat lamp and a rectal probe (Physitemp Instruments, Clifton, N.J.). Following sedation and catheterization, mice were transferred from the prep station to a custom 3D printed imaging stage, outfitted with a circulating water bath (T/Pump, Stryker, Kalamazoo, Mich.), for further treatment.

3D Volume and 2D Perfusion Imaging. 3D imaging was performed by mounting a linear 15L8 transducer, equipped with the Acuson Sequoia 512 ultrasonography system (Siemens Medical Solutions, Erlangen, Germany), on a stepper motor and sweeping it across the length of the tumor in 0.2 mm increments. Non-linear contrast images were acquired following a bolus injection of 1×10⁷ MBs in a total volume of 100 μL, administered via tail vein catheterization. Data were collected and subsequently analyzed using custom LabVIEW software, where tumor boundaries were manually segmented. The resulting series of 2D images was combined in ImageJ to compute volumetric measurements (B-mode) and to map the tumor vasculature (CPS mode). As described in Wei K, et al., Quantification of Myocardial Blood Flow With Ultrasound-Induced Destruction of Microbubbles Administered as a Constant Venous Infusion, Circulation, 1998; 97: 473-83, MB perfusion conforms to the equation y=A (1−e^{βt}), where A is the relative blood volume (RBV) and β is the rate of reperfusion. Perfusion replenishment curves following a flash-destruction pulse were generated from CPS data and fitted to this form in the LabVIEW software. Quantitative measures were extracted and compared pre- (Day 0) and post-transfection (Day 3) and plotted on the same set of axes for each mouse. Statistical analysis was performed using excel using ANOVA followed by a Tukey HSD comparison between groups.

Sonopermeation In vivo Using Focused Ultrasound Application. The image-guided sonopermeation procedure is described in Bellary A, Villarreal A, Eslami R, et al. Perfusion-guided sonopermeation of neuroblastoma: a novel strategy for monitoring and predicting liposomal doxorubicin uptake in vivo, Theranostics, 2020; 10: 8143-61. Briefly, a custom lens and cone system was 3D printed and affixed to a therapeutic ultrasound machine (SoundCare Plus, Austin, Tex.) to attain a maximum pressure of ˜2 MPa in the focal zone. A commercial infusion pump (Kent Scientific, Torrington, Conn.) was coupled to a custom 3D printed rotating syringe platform, designed to evenly disperse MBs in solution, ensuring that injections were dispensed at a fixed concentration throughout the duration of MB administration. On the day of transfection (Day 0), 1×10⁹ cationic MBs were combined with 500 μg of pDNA (either mKate or iNOS) and brought up to a total volume of 500 μL with sterile saline. The MB mixture was infused into the tumor space at a constant rate of 50 μL/min and the tumors were sonopermeated by hand (3 W/cm², 1 MHz, 10% duty cycle) on/off in intervals of 5 s over a period of 10 min. Post-sonopermeation, mice were checked daily to ensure that tumor burden did not exceed the euthanasia criteria (>2-g weight) delineated in our IACUC protocol and were further evaluated for any behavioral deficits related to pain or distress.

On the day of chemotherapeutic treatment (Day 3), 1×10⁹ regular MBs were combined with 1 mg/kg of L-DOX (Doxoves©, FormuMax, Sunnyvale, Calif.) and brought up to a total volume of 500 μL with sterile saline. The tumors were again hand scanned in the same manner as was done 72 h prior.

Animal Survival Studies. Survival in vivo experiments were performed as detailed above. Briefly, mature tumors were primed with gene therapy (either mKate or iNOS), transfected by sonopermeation with 1×10⁹ cationic polydispersed MBs having median diameter ˜2 μm. 72 h following gene therapy treatment, sonopermeation was performed using net-neutral lipid MBs to deliver liposomal doxorubicin as described above. For this experiment, due to the volume of bubbles needed, size-isolated microbubbles (SIMBs) were obtained from Advanced Microbubbles Inc to perform imaging and sonopermeation. Tumors were measured every other day until they reached the endpoint criteria up to 14 days using calipers, and re-imaged with a bolus of 1×10⁷ SIMBs on Day 7 as well as re-dosed with L-DOX in conjunction with sonopermeation. Normalized tumor growth over the two-week observation period was obtained by dividing the volume on any given day by initial tumor volume. Kaplan-Meier curves were generated to plot the number of days it took for tumors to increase by 50% above their starting volume up to 14 days. Survival curves were plotted, and statistical analyses were conducted in Graphpad (Prism 6). Statistical significance between groups represents Mantel-Cox test, with p<0.05 interpreted as significant.

Tumor Excision. Mice were sacrificed 24 h post-chemo administration (Day 4 following transfection) by exsanguination to eradicate the drug remaining in circulation. Before exsanguination, the mice were anesthetized using 5% isoflurane. After verifying the depth of anesthesia by toe pinch, the animals were catheterized and injected with a lectin stain (DyLight 594-LEL, Vector Laboratories). The lectin was allowed to circulate for 3 min, following which the mice were perfused by intracardiac injection of cold saline. This procedure was performed by inserting a syringe with 10 mL solution into the left ventricle of the heart and snipping the right atrium to allow blood to drain following a full flush of the mouse's circulatory system. All tumors were surgically excised for ex vivo processing immediately after perfusion.

Tumoral Hypoxia Measurements. 60 mg/kg pimonidazole-HCl (Hypoxyprobe, Massachusetts) was injected intraperitoneally 30 min before sacrifice, and tissues were harvested and processed as described above.

Doxorubicin Quantification in NGP Tumors. To quantify doxorubicin in excised tumors, two protocols were adapted and merged. Following excision, the tumors were weighed and flash frozen. Tissue chunks (typically 200-400 mg) were placed in 1.5 mL centrifuge tubes with a cell lysis buffer (consisting of 0.25 M sucrose, 5 mM Tris-HCl, 1 mM MgSO₄, 1 mM CaCl₂ pH 7.6) and 100 μL, ceramic beads (MO BIO Laboratories, Carlsbad, Calif.). The tubes were vortexed (Bristol-Meyers Squibb, New York, N.Y.) for 45 s to homogenize the tissue. To establish standards of known doxorubicin measurements in homogenates of tumors, untreated tissue was mixed with 2 μL of 10 mg/mL doxorubicin (Sigma Aldrich, St. Louis, Mo.) stock dissolved in DMSO and homogenized as described above. Spiked homogenates were then serially diluted with extraction buffer. The readings of untreated tumor samples without doxorubicin were considered zero. Untreated and treated homogenized samples (200 μL) were placed in microcentrifuge tubes, with acidified isopropanol solution: 100 μL of 10% (v/v) Triton X-100 (Sigma Aldrich), 200 μL of water, and 1 mL of acidified isopropanol (0.75 N HCl, Sigma Aldrich). Samples were stored overnight at −20° C. to extract the doxorubicin. The next day, the tubes were warmed to room temperature, vortexed for 45 s, centrifuged at 2,000 g for 15 min and stored at −80° C. until analysis.

A five-point standard curve generated by spiking tissue with known quantities of doxorubicin was run side by side with experimental samples to quantify the uptake per gram of tumor using linear regression. Statistical analysis was performed using excel using ANOVA followed by a Tukey HSD comparison between groups.

Immunohistochemistry. Excised NGP tumors were embedded in Tissue-Tek® optimum cutting temperature (O.C.T.) compound (Electron Microscopy Services), then stored at −20 ° C. until cryosectioned (Leica CM1860). 15 μm thick cryosections were fixed with acetone and permeabilized with Tween 20. Following blocking with CAS-Block Histochemical Reagent (ThermoFisher Scientific), the following primary antibodies were used: murine iNOS (1:500, #13120, Cell Signaling), aSMA-Cy3 (1:1000, #C6198, Sigma), pimonidazole (1:100, #Pab2627, Omnikit, Hypoxyprobe, Massachusetts). Isolectin-B4-AF568 (1:100, #121412, Invitrogen), was diluted in HEPES buffer. Alexa Fluor 488 goat anti-rabbit IgG (Invitrogen) secondary antibody was used. Finally, the slides were mounted with DAPI (VECTASHIELD PLUS Antifade Mounting Medium with DAPI, Vector Laboratories).

Quantitative Ex Vivo Imaging. For iNOS expression colocalization studies, sections were imaged on a Marianas Confocal (Zeiss) using a 40X oil objective, capturing 14 steps of 0.33 μm on the z-axis, with a resolution of 0.33 μm per pixel. At least five images of each tumor of were taken, with four tumors per group. To avoid bias, the endothelial marker Isolectin-B4 was used to determine image capture and focus and iNOS staining was captured at equal exposure times for all tissues. The images were then analyzed in ImageJ (NIH, USA), selecting the lectin-positive area of the picture taken to create the area to be quantified. This area was then used to quantify the mean intensity of the iNOS staining within the tumor endothelium. The mean iNOS intensity within each tumor endothelium z-stack was then averaged to obtain the mean iNOS intensity of each tumor blood vessel analyzed. Averages were then obtained for each tumor. Because tumor cells were injected directly into the kidney and often coopt glomeruli and tubules (PMID: 24066611), NOS expression was quantified in the adjacent kidney in a separate analysis, following the same method as tumor iNOS quantification. Quantification of pimonidazole and Isolectin was performed using the average intensities from the respective stains comparing the iNOS treated and Sham groups using FIJI (NIH). Terminal deoxynucleotidyl transferase dUTP nick end labeling (TUNEL) staining was performed on fresh frozen sections following the manufacturer's instructions (Millipore, USA), and was visualized on a whole slide scanner (Olympus VS120 Virtual Slide Microscope) at 447 nm (DAPI), 510 nm (FITC), and 624 nm (Texas Red) laser wavelengths.

The distance of saSMA from lectin was measured using the distance tool on scans using the Olympus VS120 software tools. 25 measurements were taken from each tissue section and the averages of three sections per group were then used to calculate statistical differences using Student t-test (GraphPad Prism), with the significance threshold set at p<0.05.

### RESULTS

### Low-Dose L-DOX Synergizes with iNOS Overexpression in NGP Cells In Vitro

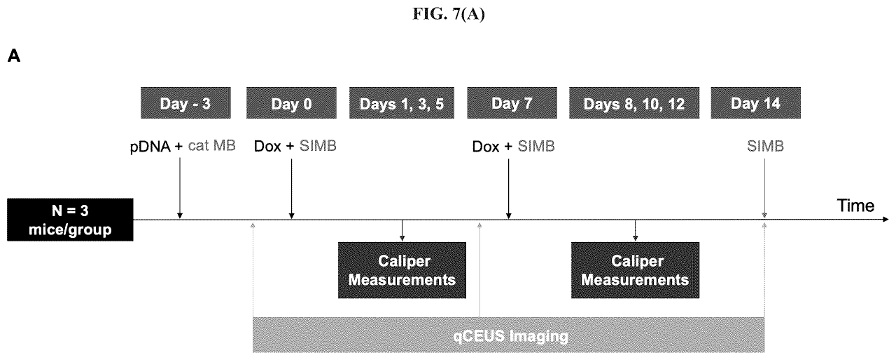

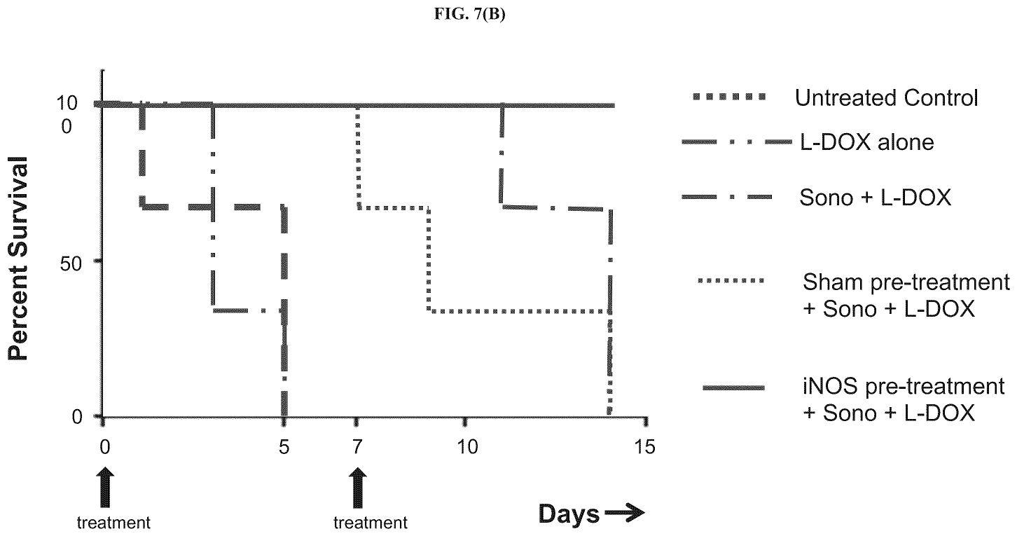

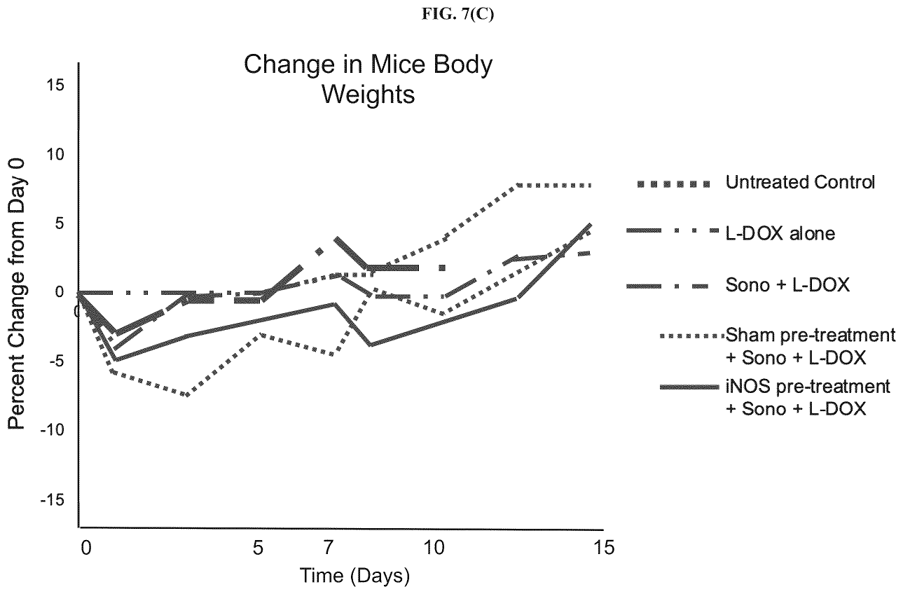

Multiple studies have shown that iNOS alone can have adverse effects on multiple tumor cells in vitro (PMID: 1382130, PMID: 7509718, PMID: 7541238). To evaluate the iNOS interaction with L-DOX in NGP cells, NGP cell proliferation was assessed after transient iNOS transfection with increasing L-DOX concentrations (0, 5 and 10 mM). In the absence of drug, iNOS transfection alone reduced proliferation by 15% compared to Sham (FIG. 2B, Sham vs 0.49±0.005 iNOS, absorbance units, p<0.01). In the Sham group, low L-DOX concentrations (5 mM) resulted in a significant albeit small (7%) reduction in cell numbers compared to no L-DOX (FIG. 7D). In comparison, iNOS sensitized NGP cells to 5 mM L-DOX with a 22% reduction in cell number relative to untreated iNOS cells (0.49±0.03 no L-DOX vs. low L-DOX, absorbance units, p<0.001). This interaction did not extend to higher L-DOX doses, however, as iNOS transfected cells receiving either 5 or 10 mM L-DOX were not different from each other (0.38±0.03 low L-DOX vs. 0.35±0.035 high L-DOX, absorbance units, p=0.17). This suggests that iNOS sensitizes NGP cells to L-DOX only in lower L-DOX concentrations. Protein from NGP cells transfected and harvested at the time of cell proliferation assay indicate that iNOS transfection resulted in a 5-fold iNOS overexpression compared to Sham (FIG. 2C, 0.04 units Sham vs. 0.2±0.03 units iNOS, n=3, p<0.0001). Together, this data shows that iNOS overexpression inhibits NGP cell proliferation by 15%, and that its interaction with L-DOX accounts for a further 12% cell loss for a combined 27% reduction in cell proliferation.

### Sonopermeation Using Focused Ultrasound and Cationic Bubbles Effectively and Specifically Transfects Tumor Vascular Endothelium

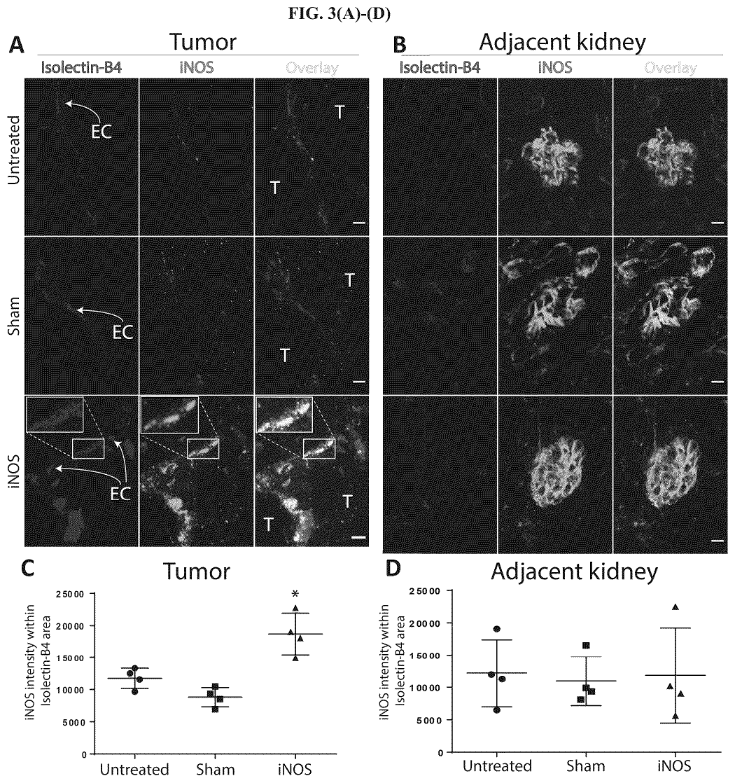

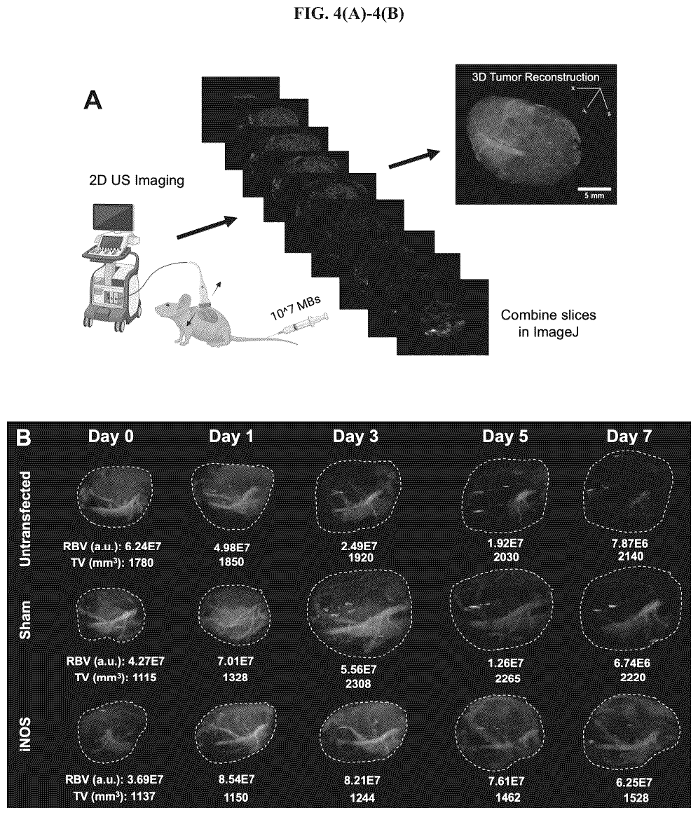

As mice were perfused with the endothelial marker Isolectin-B4 prior to euthanasia, the presence of this stain in sections represents endothelial cells of functional vasculature. FIG. 3 shows that iNOS transfection via sonopermeation increases iNOS expression in tumor endothelial cells in NGP tumor-bearing mice but not in adjacent kidney structures. FIG. 3A shows confocal microscopy images revealing elevated iNOS expression in tumor endothelium after iNOS transfection compared to untreated or Sham transfected tumors (bottom right panel and inset). EC: endothelial cells, T: tumor cells. FIG. 3B shows representative images of glomeruli (labeled “G”) adjacent to NGP tumors. FIG. 3C shows quantification of the iNOS signal intensity shows a greater than two-fold increase within the lectin-positive area in iNOS transfected tumors compared to untreated or Sham transfected tumors, indicating enhanced iNOS activity. FIG. 3D shows iNOS expression in adjacent glomeruli remains unchanged after iNOS or Sham transfections (p>0.05). Scale bar=10 μm. n=4 per group.

iNOS expression in the tumor vasculature was evaluated using Isolectin-B4 72 h after transfection to allow for maximal protein expression. FIG. 3A highlights representative images of tumors while 3B highlights representative images from the adjacent non-targeted healthy kidney tissue; both sets of images were derived from projections of z-stacks obtained with confocal microscopy. Endothelial cells are labeled “EC” in the figure and adjacent tumor cells are marked “T”. Immunohistochemistry analysis revealed a significant increase in endothelial iNOS expression in the iNOS transfected endothelial cells (FIG. 3A, lower middle and right panels), while Sham transfected tumor endothelial cells had no change in iNOS compared with untreated tumors (FIG. 3A, middle row shows no increase in iNOS, compared to untreated controls). To quantify the iNOS expression in the tumor endothelium, iNOS intensity was restricted within the lectin-positive areas (FIG. 3C). Quantification of the iNOS mean intensity within the lectin-positive area revealed over 2-fold increase in iNOS expression after iNOS transfection compared to Sham transfected tumors and over 1.5-fold compared to untreated controls (FIG. 3B, mean intensity values: 11,749±1,576 untreated, 8,814±2,952 Sham, 18,709±5,993 iNOS, p<0.01). To evaluate the specificity of iNOS sonopermeation, iNOS expression was quantified in neighboring kidney structures (FIG. 3C, glomeruli labeled “G”) using the same methodology used to quantify tumor iNOS expression. In the adjacent kidney, no change in iNOS expression was found regardless of the group, suggesting that iNOS overexpression was localized to the targeted sonopermeated tumor.

Transfection with iNOS Increases Blood Volume and Flow in Neuroblastoma Xenografts

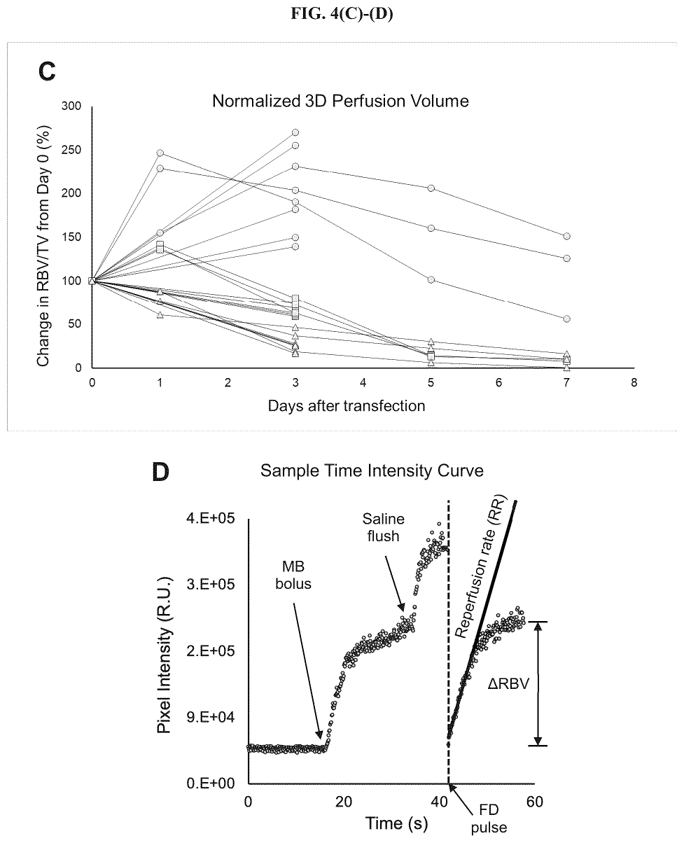

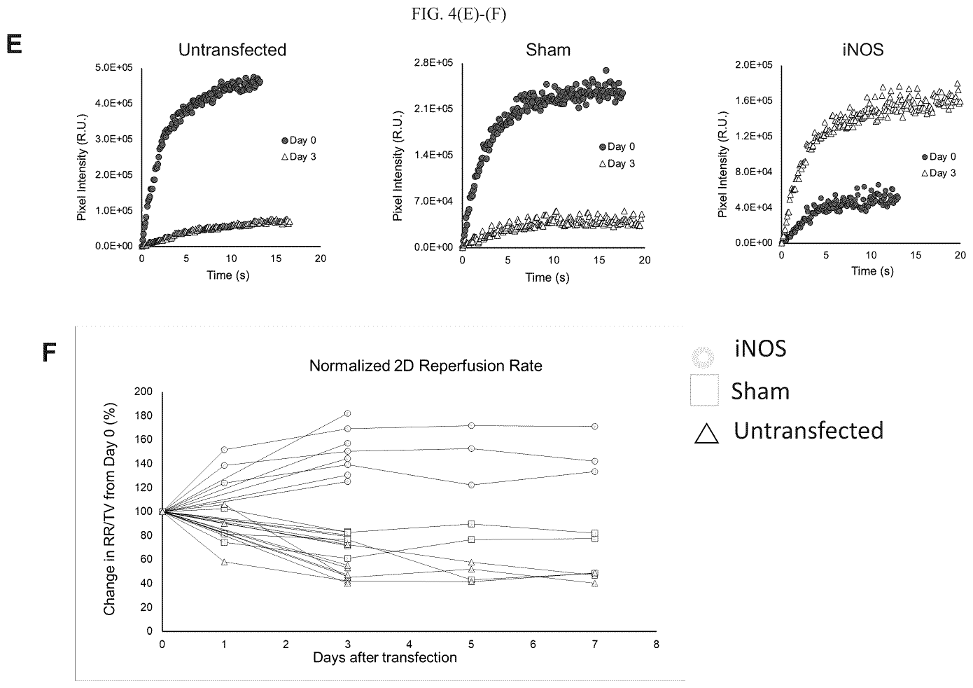

FIG. 4 relates to the longitudinal effects of iNOS transfection on NGP tumor vasculature and perfusion dynamics in a 7-day time course. FIG. 4A shows a schematic representation of the workflow for generating 3D tumor reconstructions from 2D contrast images. FIG. 4B shows representative 3D contrast images displaying the increase in perfusion-volume post-treatment in comparison with Sham-treated and untreated controls. FIG. 4C shows normalized tumor perfusion volumes are plotted over a 7-day period, demonstrating a significant increase in perfusion post-treatment. FIG. 4D shows time-intensity curves derived from non-linear 2D imaging to assess perfusion dynamics in NB tumors. FIG. 4E shows flash-destruction (FD) replenishment curves reveal augmented perfusion rates in iNOS-treated tumors compared to controls. FIG. 4F shows quantitative analysis of normalized tumor 2D re-perfusion rates after FD indicating enhanced perfusion in response to iNOS treatment over the 7-day time course. n=7-8 mice per group. * indicates p<0.05.

Quantitative contrast-enhanced imaging was performed at 0 (before transfection) and 3 days post transfection. Several mice from each treatment group were also monitored on days 1, 5, and 7. Perfusion volume was gleaned from whole tumor 3D reconstructions (FIG. 4A) and flow rates were extracted from 2D MB time-intensity curves (TICs) following a flash-destruction pulse (FIG. 4D). Blood perfusion volume (RBV) was determined in LabVIEW by summing pixel intensities throughout the tumor volume following an intravenous injection of MBs. Since MBs rapidly mix and circulate with blood upon systemic infusion, the overall enhancement in video signal intensity denotes the total blood pool volume in that region. Examples showing 0-3 day and 0-7 day perfusion trends are displayed in FIG. 4B. To generate a measure of the density of vessels within the tumor, RBV values were divided by tumor volume (TV) and plotted during the week after transfection (FIG. 4C). Vascularity changes were seen as early as 24 h and the change in RBV from baseline was calculated at 3 days post-transfection. iNOS tumors increased in vascularity by 213±47% while Sham and untreated controls decreased to 68±7% and 32±12% respectively. ANOVA followed by the Tukey HSD test was performed. The p-value corresponding to the F-statistic of one-way ANOVA was lower than 0.05, suggesting that the one or more treatments are significantly different. The Tukey HSD p-values for the iNOS group vs. the Sham and untreated control groups were p<0.01. The p-value between the Sham and untreated control was p=0.06, indicating that these groups were not statistically different on day 3. Interestingly, the vascularity of the Sham group did appear to increase at 24 h, consistent with previous findings, supporting the conclusion that sonopermeation increases vascular permeability and enlarges vessel lumens (reversibly) for up to one day post-treatment.

The rate of MB reperfusion (RR), which is representative of blood flow, was normalized to TV and monitored from 0-3 days or over a one-week period (FIG. 4F). Flow rates improved drastically 3 days after iNOS treatment (150±18%) and remained at increased levels over the next few days. Sham-treated mice and untreated control mice both showed lower reperfusion at day 3 (77±8% and 50±10% respectively) and continued to decrease over 7 days. ANOVA followed by the Tukey HSD test was performed. The p-value corresponding to the F-statistic of one-way ANOVA was lower than 0.05, suggesting that the one or more treatments are significantly different. The Tukey HSD p-values for the iNOS group vs. the sham and untreated control groups were p<0.01 and the Sham vs. untreated control group had a p<0.01, indicating that all groups were significantly different.

Transfection with iNOS Increases Perfusion in Neuroblastoma

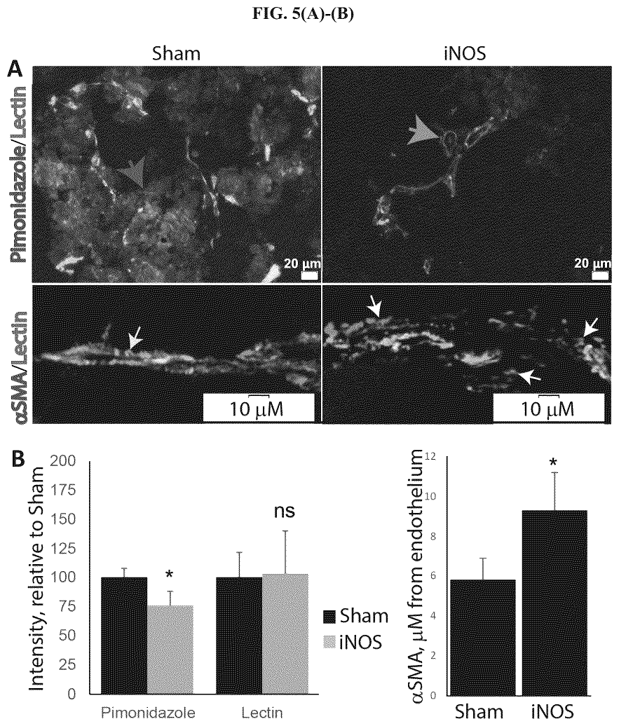

FIG. 5 shows that iNOS transfection increases tumoral perfusion by remodeling tumor vasculature and reducing tumor hypoxia. FIG. 5A shows that iNOS transfection reduced tumor hypoxia as indicated by a decrease in pimonidazole immunoreactivity relative to Sham-transfected tumors. Increased vascular diameter or lumens was evident. aSMA-positive pericytes were located further away from lectin-positive endothelial cells in iNOS-transfected tumors, than Sham tumors with adjacent or overlapping pericyte coverage of endothelium. FIG. 5B shows quantification of pimonidazole and lectin stains (left) demonstrates decreased hypoxia in iNOS tumors. Pericyte distance from the endothelium increased as a result of iNOS transfection. n=5 mice per group. * indicates statistical significance (p<0.05) in the amount of hypoxia and pericyte quantifications.

Changes in vascularity post-transfection were interrogated using immunohistochemical analysis on tumor tissues harvested 72 h after transfection. The endothelial marker, Isolectin-B4, revealed no change in the total amount of endothelial cells, but an increase in the vascular lumen was evident, consistent with NO vasodilation effects (FIG. 5A), suggesting that angiogenesis is not occurring because of iNOS transfection. In line with the pericyte relaxation effects of NO, the distance between alpha smooth muscle actin (aSMA) pericytes and endothelium was 60% higher in iNOS-transfected tumors compared to the Sham group (FIG. 5B, 5.8±1.1 mm Sham vs. 9.3±1.9 mm iNOS, p<0.05). An example of pericyte distance from endothelium is depicted in FIG. 5A (lower panel), unlike Sham tumors where pericytes are either directly adjacent or overlap with the endothelium. To gauge hypoxia, pimonidazole injected 30 min before sacrifice was quantified; staining revealed a 24±12% (p<0.05) decrease in the amount of pimonidazole in the iNOS-transfected tumors compared to the Sham group (FIG. 5A and FIG. 3B, graph). Together, these findings demonstrate a sustained tumoral vasodilation that increases blood volume, along with decreased pericyte support of endothelial cells resulting in increased permeability and reduced intratumoral hypoxia levels.

iNOS Transfection Increases Doxorubicin Uptake in Orthotopic NB Xenograft Tumors and Increases Apoptosis

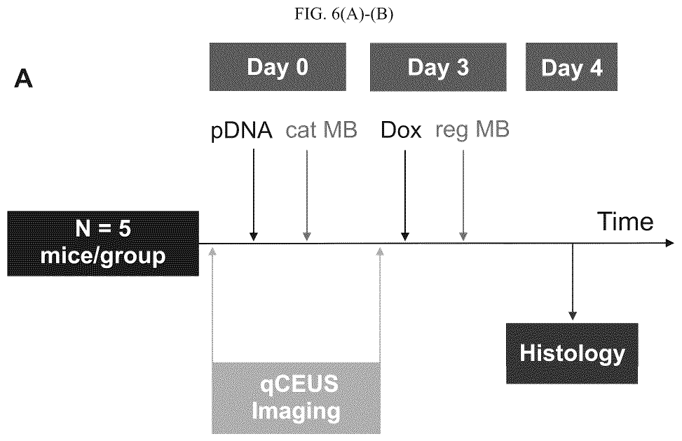

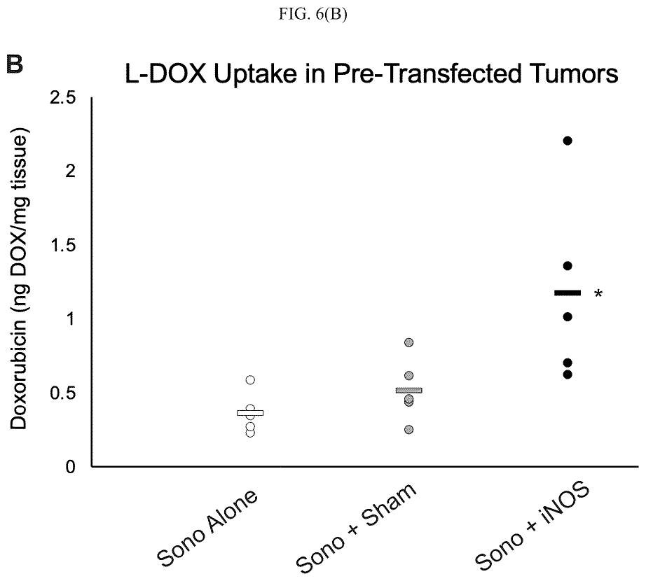

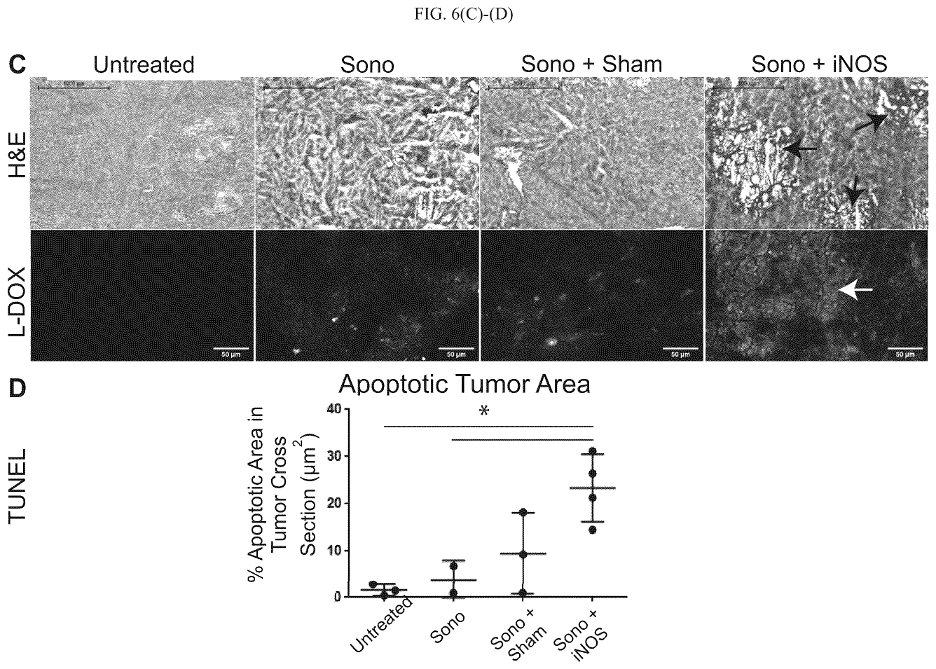

FIG. 6 shows effect of pre-treatment with iNOS gene therapy on doxorubicin uptake. FIG. 6A shows a summary of the methodology for priming tumors with iNOS before treatment. qCEUS imaging was performed using a bolus of 1×10⁷ regular MBs before sonopermeating on days 0 and 3. Tumors were excised and processed for ex vivo analysis 24 h post-chemo. FIG. 6B shows that pretreatment of tumors with iNOS significantly increased drug uptake, as demonstrated by a 3.3-fold increase in doxorubicin accumulation compared to untransfected counterparts, and 2.3-fold boost compared to tumors receiving Sham treatment. FIG. 6C shows representative images of doxorubicin uptake revealing that Sham tumors had slightly higher uptake compared to controls, while iNOS tumors had significantly higher uptake than both groups (top right panel). Similarly, examination of the apoptosis marker TUNEL showed increased staining in proportion to doxorubicin uptake, with iNOS transfection resulting in the largest areas of TUNEL staining (bottom right panel). FIG. 6D shows that quantification of TUNEL areas normalized by total tumor area showed that iNOS-transfected tumors had significantly higher levels of apoptosis compared to untreated controls receiving L-DOX without sonopermeation or untransfected tumors given L-DOX with sonopermeation (p<0.05). Furthermore, this level approached significance compared to Sham tumors (p=0.08). n=5 mice per group in doxorubicin extraction and n=3-4 in histological analysis. * indicates statistical significance (p<0.05) with respect to all other groups.

To investigate whether priming with iNOS expression enhances chemotherapeutic uptake, NGP tumors were sonopermeated with 10⁹ net-neutral MBs together with 1 mg/kg L-DOX 3 days post-transfection (FIG. 6A). Tumors pretreated with iNOS plasmids before L-DOX sonopermeation had a 327±64% increase in doxorubicin fluorescent intensity compared to untransfected tumors receiving L-DOX sonopermeation and 228±39% doxorubicin fluorescent intensity compared to tumors pretreated with Sham plasmid (FIG. 6B). Untreated controls (no sonopermeation, L-DOX alone) were used to measure the baseline fluorescence intensity for comparison. The p-value corresponding to the F-statistic of one-way ANOVA was lower than 0.05, suggesting that the one or more treatments are significantly different. The Tukey HSD test comparing the treatment groups showed that the iNOS was significantly higher than both the sham and untreated control groups (p<0.05), but tumors pre-treated with Sham plasmids and untransfected tumors receiving L-DOX sonopermeation were not statistically different (p=0.82) These changes in doxorubicin quantification were mirrored qualitatively by microscopy (FIG. 6C, top panels). Finally, the percentage of areas positive for the apoptosis marker TUNEL was quantified and a higher degree of apoptosis in L-DOX sonopermeated tumors pretreated with iNOS was confirmed compared to untreated controls receiving L-DOX without sonopermeation (p<0.01) and untransfected tumors sonopermeated with L-DOX (p=0.02) (FIG. 6C, lower panel, and 6D quantification). Although the apoptosis area in the Sham group was not significantly different from iNOS tumors (p=0.08), iNOS pretreated tumors had large non-viable areas that likely led to the undercounting of apoptosis in this group only. Jointly, these data suggest that pre-treatment with iNOS augments tumoral perfusion to the extent that higher drug payloads amass in the tumor, even when L-DOX is administered at very low doses.

iNOS Transfection Followed By L-DOX Increases Median Survival Time

FIG. 7 shows the effect of pre-treatment with iNOS gene therapy on tumor growth. FIG. 7A shows a summary of the procedure adopted to prime tumors with gene therapy before low dosage chemotherapy plus sonopermeation; mice were re-dosed at day 7 and monitored for 14 days or until the endpoint criteria were met. qCEUS imaging was performed using a bolus of 1×10⁷ size-isolated MBs before sonopermeating on days 0, 7, and 14. FIG. 7B shows that iNOS transfection significantly increases survival time in NGP tumors. FIG. 7C shows the change in percentage body weight for each treatment group. n=3 mice per group.

To understand the relationship between iNOS-mediated perfusion increases and volumetric tumor growth, two-week studies in which mice were dosed and re-dosed with L-DOX along with sonopermeation on days 0 and 7 were undertaken with and without pre-transfection. Mice with no treatment had a median survival time of 5 days (FIG. 7B), while mice receiving only L-DOX took a median of 3 days to increase in volume by 50% above baseline (FIG. 7C) (p=ns). In contrast, sonopermeation alone (FIG. 7B) followed by L-DOX resulted in a median survival time of 14 days, while Sham plasmid transfection (FIG. 7B) followed by L-DOX resulted in a median survival time of 9 days. While Sham and sonopermeation alone were not statistically different from each other (p=ns), both took significantly longer to reach 50% growth than untreated and L-DOX only mice (p<0.05). Finally, none of the mice receiving iNOS transfection followed by L-DOX reached 50% tumor growth after 14 days, defined as the endpoint of the study (FIG. 7C), thus surviving significantly longer than mice in any other group (p<0.05). This longitudinal data shows that iNOS transfection combined with L-DOX treatment prolongs survival time.

### Discussion

For the past three decades, passive targeting by the Enhanced Permeability and Retention (EPR) effect has had uneven success by making use of nanoscale vehicles, such as liposomes, to shuttle cargo into tumors. While it can be effective (particularly for long-circulating liposomes), mounting evidence alludes to its highly variable nature, rather than a generalizable phenomenon as it was once regarded. Moreover, 50-60% of solid tumors have a low EPR environment, making them exceedingly hard to target. These cancers, including NB, tend to be hypoxic and poorly vascularized, and in many cases even coopt vessels from nearby healthy tissues, which are not inherently leaky. The lack of fenestration and poor perfusion can be significant obstacles to drug penetration in tumor therapy.

Image-guided drug delivery (IGDD) using shows that tumor perfusion volume serves as a robust predictor of drug accumulation. Doxorubicin uptake in sonopermeated tumors correlates positively with perfusion volume, indicating that the initial degree of vascularity greatly influences the extent to which sonopermeation enhances drug uptake. Pursuing NO-based gene therapy improves tumor perfusion and allows for increased drug penetration. The present methods successfully employed a targeted non-viral gene therapy strategy to modify NB biology to improve the efficacy of a chemotherapeutic drug.

Non-viral therapies are well known for having low transgene production compared to viral vectors. Transfection of tumors in vivo using sonopermeation increases iNOS expression in the targeted tumor vasculature. However, high levels of transgene expression may not be required for effective NO therapy when using an efficient producer of this molecule. The methods described herein use delivery of a plasmid DNA (pDNA) encoding a functional copy of an inducible variant of the NOS enzyme, so that NO can be produced by catalytic conversion of L-arginine, independent of calcium/calmodulin signaling. Multiple isoforms of NOS exist throughout the body with varying levels of NO production. Inducible NOS (iNOS) is the most potent isoform that can generate several orders of magnitude more NO (100 to 1000-fold greater) than constitutive endothelial or neuronal NOS (eNOS and nNOS), until substrate availability becomes rate-limiting. Several studies have highlighted the potential role of iNOS in cancer therapy. The current methods leverage selective iNOS transfection of the tumor vascular endothelium, thus narrowing its effect on the tumor vasculature and limiting its potential effects on tumor cells. Thus, iNOS is exploited as a means of modulating intratumoral vascular NO production, thereby enhancing tumor perfusion spatiotemporally.

This present methods also advance gene therapy efficacy with the use of a platform that merges ultrasound-triggered cell-entry and enhanced gene transfection. The status quo as it pertains to gene delivery is that non-viral vectors have not been widely adopted to address major in vivo barriers: (1) degradation of the therapeutic genes by endonucleases in the serum, (2) selective deposition of therapeutic genes into the tumor tissue, and (3) plasmid internalization into tumor or tumor vascular cells. By incorporating 20% molar mass of the cationic phospholipid DOTAP into the microbubble shell, they are capable of electrostatically binding negatively charged plasmids and protecting them from degradation in circulation. When orthotopic NGP xenografts were locally transfected by applying FUS during a systemic administration of pDNA-bound bubbles, iNOS was detected post-transfection by IHC, with high levels of the expressed protein product (iNOS) present 72 h after transfection (FIG. 3A). Sonopermeation using a Sham plasmid increased iNOS expression as well. However, most of this upregulation could be found in coopted kidney structures rather than the tumor itself. Upregulation of iNOS in inflamed and damaged kidney is consistent with previous literature. iNOS overexpression within the tumor required functional iNOS-expressing plasmids (FIG. 3B). Earlier research demonstrated that the gene expression occurs only in areas where focused ultrasound is applied, therefore effects of systemic treatment can be confined strictly to tumor tissue. These data suggest that sonopermeation is a highly efficient alternative to virus-mediated delivery, which is the current gold standard for gene therapy.

Regarding whether whole tumor perfusion was affected by iNOS overexpression, perfusion decreased at 3 days in untreated controls and in tumors transfected with an identical plasmid expressing a non-functional protein, mKate (“Sham”), as compared with those transfected with iNOS in which perfusion volume increased significantly (FIG. 4). The sample 3D tumor reconstructions showcased in FIG. 4B exemplify the global changes that are encapsulated graphically in FIG. 4C: hypoxic pockets adjacent to the tumor vasculature exist in the untransfected tumors, suggesting nonfunctional vasculature, while such unperfused regions are less pervasive in the iNOS tumors. In contrast, tumors that are transfected with iNOS showed significant increases in perfusion volume within the tumor space over time (FIG. 4C). This suggests that iNOS may be responsible for pericyte relaxation, as suggested in FIG. 5A, and thus promotes vessel enlargement. NO-dependent diversion of O₂ may reactivate enzymes whose functionalities are diminished by hypoxia, signifying that treatment with iNOS may delay the emergence of hypoxic conditions within tumors. Indeed, the hypoxia marker pimonidazole revealed iNOS expression decreased hypoxia compared to Sham (FIG. 5A). Analysis of the vasculature showed widened vascular lumens and increased pericyte distance from the lumen because of iNOS expression, in agreement with its known effect as a vasodilator and its effects on pericyte relaxation.

The biological effects of NO therapy can be highly variable and are highly dependent on local NO concentration in tissue. It is not feasible to monitor NO concentrations directly since its half-life in vivo is <2 ms. Consequently, it was critical to observe the effects of NO therapy in real-time to identify when the tumors became the most susceptible to treatment. Examining qCEUS parameters offers evidence of iNOS' vascular effects: whereas untransfected and Sham tumors experience a decrease in MB flow rates (FIG. 4F), iNOS tumors show increases in both the rate of reperfusion (RR) and relative blood volume (RBV). In FIG. 4E, superimposed pre- and post-transfection reperfusion curves reveal that declining RR and RBV trends in the Sham and untreated control groups are entirely reversed in the iNOS treatment group; ex vivo analysis of these tumors divulges that iNOS samples present with significantly dilated vessel lumens (FIG. 5). Monitoring of the tumors post-transfection using qCEUS imaging can therefore be used to identify optimal windows of therapy for treatment in a clinical setting.

Doxorubicin is an integral part of NB standard-of-care. However, cardiotoxicity associated with high dosage chemotherapy is a significant clinical problem. Liposomal doxorubicin (L-DOX) can be efficiently delivered using sonopermeation. This form of doxorubicin, which is clinically available, has a much longer circulation lifetime and reduced cardiotoxicity profile. The effects of iNOS gene therapy was tested on prime tumors for liposomal drug treatments. L-DOX was administered along with sonopermeation 72 h after iNOS or Sham transfection and amplified drug accumulation was quantified in iNOS-expressing tumors ex vivo, both by tissue extraction and histology (FIG. 6). The increase in tumoral L-DOX uptake was confirmed by DOX detection in tumoral tissue samples and a corresponding increase in tumor apoptosis quantification (FIG. 6). Tumor integrity was severely compromised in several of the iNOS-transfected tumors treated with L-DOX and sonopermeation, which likely led to an underestimation of tumor apoptosis in iNOS-transfected tumors during quantification, further strengthening our findings. Heightened L-DOX uptake and apoptosis in iNOS-transfected tumors (FIG. 6) also correlated with increased tumoral survival time (FIG. 7). In fact, all the mice receiving iNOS transfection and low-dose L-DOX had no tumor growth at the end of the study, supporting the efficacy of this approach.

To parse out the contribution of iNOS expressed in tumor cells versus vasculature and its potential interaction with L-DOX, iNOS was overexpressed in cultured tumor cells and increasing L-DOX concentrations were added. Overexpressing iNOS inhibited tumor cell proliferation and synergized with lower L-DOX concentrations (FIG. 2). This interaction, however, was not observed with higher L-DOX concentrations. The data is consistent with previous studies demonstrating that iNOS enhances cancer cell chemotoxicity and radiotoxicity in vitro and in vivo. Possible mechanisms for L-DOX interaction with iNOS include reactive oxygen species, caspases, p53, and NF-kB. Together, the data suggests that iNOS overexpression in tumor cells sensitizes them to the low-dose L-DOX therapy, contributing to the enhanced survival of animals receiving iNOS transfection in conjunction with low doses of L-DOX.

The L-DOX dosage used (1 mg/kg) is commensurate with a human equivalent dosage that is -25 times below the standard-of-care in high-risk neuroblastoma therapy. In previous studies when three doses of thermosensitive liposomes encapsulating doxorubicin (LTLD) (0.1, 0.5, and 2.5 mg/kg) were dispensed together with MR-guided high-intensity focused ultrasound (MR-HIFU) induced hyperthermia in a rabbit Vx2 tumor model, it was observed that lower DOX uptake efficiencies, defined as the ratio of the accumulated tissue DOX concentration to the injected dose, correlated with higher overall doses. It was speculated that diminishing returns occur with increasing DOX doses, possibly due to intracellular uptake saturating at higher extracellular concentrations, in agreement with earlier reports using a dosage of 5 mg/kg. These data suggest that low-dosage chemotherapies can be applied with sonopermeation to augment the current standard-of-care without conferring an increased risk of systemic toxicity. Furthermore, this strategy help reduce dosages to cut down on off-target accumulation in intermediate risk NB where micrometastasis has not occurred. Higher tumoral doxorubicin concentrations and pro-apoptotic effects can be achieved at low drug doses after iNOS transfection, thus constraining the potential side effects of chemotherapy drugs. This drug deposition pattern is not exclusive to doxorubicin, and can be accomplished with other chemotherapy agents and solid tumor models.

As shown herein, pre-treatment with iNOS is a clinically viable solution to improve NB response to chemotherapy. Additionally, elevated intratumoral endothelial iNOS has been linked with radiosensitivity, supporting that the present methods can also make tumors more amenable to radiotherapy as well as standard-of-care chemotherapies.

What is claimed is:

1. A method for administering a therapeutic agent to a tumor in a subject, comprising:
loading iNOS-expressing plasmid DNA into microbubbles to produce loaded microbubbles;
infusing the loaded microbubbles into a space surrounding the tumor in the subject;
applying image-guided focused ultrasound to the tumor, whereby the iNOS-expressing plasmid DNA is delivered selectively into the tumor through sonopermeation, whereby iNOS is selectively expressed in the tumor, and whereby nitric oxide levels in the tumor increase;
allowing perfusion of the tumor to increase over a period of time as a result of increased nitric oxide levels; and
administering a therapeutic agent to the space surrounding the tumor in the subject, whereby uptake of the therapeutic agent into the tumor occurs.

2. The method of claim 1, further comprising a step of monitoring perfusion rate of the tumor to identify an optimal time for administering the therapeutic agent, prior to the step of administering the therapeutic agent to the space surrounding the tumor in the subject.

3. The method of claim 2, wherein the step of monitoring tumor perfusion rate is by using longitudinal quantitative contrast-enhanced ultrasound imaging.

4. The method of claim 3, wherein the longitudinal quantitative contrast-enhanced ultrasound imaging visualizes circulation of microbubbles in blood vessels of the tumor and the space surrounding the tumor in the subject.

5. The method of claim 2, wherein the optimal time for administering the therapeutic agent is a time when the tumor perfusion rate is increased.

6. The method of claim 1, wherein the step of administering the therapeutic agent to the space surrounding the tumor in the subject comprises loading the therapeutic agent into microbubbles to produce therapeutic loaded microbubbles, infusing the therapeutic loaded microbubbles into the space surrounding the tumor in the subject, and applying image-guided focused ultrasound to the tumor, whereby the therapeutic agent is delivered selectively into the tumor through sonopermeation.

7. The method of claim 1, wherein the therapeutic agent is a liposome-encapsulated chemotherapy drug.

8. The method of claim 1, wherein the tumor is caused by cancer.

9. The method of claim 6, wherein the cancer is neuroblastoma.

10. A method for treating neuroblastoma in a subject, comprising:
loading iNOS-expressing plasmid DNA into microbubbles to produce loaded microbubbles;
infusing the loaded microbubbles into a space surrounding a tumor in the subject, wherein the tumor is caused by neuroblastoma;
applying image-guided focused ultrasound to the tumor, whereby the iNOS-expressing plasmid DNA is delivered selectively into the tumor through sonopermeation, whereby iNOS is selectively expressed in the tumor, and whereby nitric oxide levels in the tumor increase;
allowing perfusion of the tumor to increase over a period of time as a result of increased nitric oxide levels;
monitoring perfusion rate of the tumor using longitudinal quantitative contrast-enhanced ultrasound imaging to identify an optimal time for administering the therapeutic agent, wherein the longitudinal quantitative contrast-enhanced ultrasound imaging visualizes circulation of microbubbles in blood vessels of the tumor and the space surrounding the tumor in the subject, and wherein the optimal time for administering the therapeutic agent is a time when the tumor perfusion rate is increased;
loading a liposome-encapsulated chemotherapy drug into microbubbles to produce therapeutic loaded microbubbles;
infusing the therapeutic loaded microbubbles into the space surrounding the tumor in the subject; and
applying image-guided focused ultrasound to the tumor, whereby the liposome-encapsulated chemotherapy drug is delivered selectively into the tumor through sonopermeation, and whereby the liposome-encapsulated chemotherapy drug increases apoptosis in the tumor and treats the neuroblastoma in the subject.


SEQUENCE LISTING (3 sequences)

SEQ ID NO: 1 - DNA, 37 bp, synthetic construct
  source 1..37: mol_type = other DNA
cagtagaccggtgagactctggccccacgggacacag

SEQ ID NO: 2 - DNA, 37 bp, synthetic construct
  source 1..37: mol_type = other DNA
cagtagcaattggaattgtaatacgactcactatagg

SEQ ID NO: 3 - DNA, 9553 bp, synthetic construct
  source 1..9553: mol_type = other DNA
ggccttttgctggccttttgctcacatgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggctccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgagactctggccccacgggacacagtgtcactggtttgaaacttctcagccaccttggtgaagggactgagctgttagagacacttctgaggctcctcacgcttgggtcttgttcactccacggagtagcctagtcaactgcaagagaacggagaacgttggatttggagcagaagtgcaaagtctcagacatggcttgcccctggaagtttctcttcaaagtcaaatcctaccaaagtgacctgaaagaggaaaaggacattaacaacaacgtgaagaaaaccccttgtgctgttctcagcccaacaatacaagatgaccctaagagtcaccaaaatggctccccgcagctcctcactgggacagcacagaatgttccagaatccctggacaagctgcatgtgacatcgacccgtccacagtatgtgaggatcaaaaactggggcagtggagagattttgcatgacactcttcaccacaaggccacatcggatttcacttgcaagtccaagtcttgcttggggtccatcatgaaccccaagagtttgaccagaggacccagagacaagcctacccctctggaggagctcctgcctcatgccattgagttcatcaaccagtattatggctcctttaaagaggcaaaaatagaggaacatctggccaggctggaagctgtaacaaaggaaatagaaacaacaggaacctaccagctcactctggatgagctcatctttgccaccaagatggcctggaggaatgcccctcgctgcatcggcaggatccagtggtccaacctgcaggtctttgacgctcggaactgtagcacagcacaggaaatgtttcagcacatctgcagacacatactttatgccaccaacaatggcaacatcaggtcggccatcactgtgttcccccagcggagtgacggcaaacatgacttcaggctctggaattcacagctcatccggtacgctggctaccagatgcccgatggcaccatcagaggggatgctgccaccttggagttcacccagttgtgcatcgacctaggctggaagccccgctatggccgctttgatgtgctgcctctggtcttgcaagctgatggtcaagatccagaggtctttgaaatccctcctgatcttgtgttggaggtgaccatggagcatcccaagtacgagtggttccaggagctcgggttgaagtggtatgcactgcctgccgtggccaacatgctactggaggtgggtggcctcgaattcccagcctgccccttcaatggttggtacatgggcaccgagattggagttcgagacttctgtgacacacagcgctacaacatcctggaggaagtgggccgaaggatgggcctggagacccacacactggcctccctctggaaagaccgggctgtcacggagatcaatgtggctgtgctccatagtttccagaagcagaatgtgaccatcatggaccaccacacagcctcagagtccttcatgaagcacatgcagaatgagtaccgggcccgtggaggctgcccggcagactggatttggctggtccctccagtgtctgggagcatcacccctgtgttccaccaggagatgttgaactatgtcctatctccattctactactaccagatcgagccctggaagacccacatctggcagaatgagaagctgaggcccaggaggagagagatccgatttagagtcttggtgaaagtggtgttctttgcttccatgctaatgcgaaaggtcatggcttcacgggtcagagccacagtcctctttgctactgagacagggaagtctgaagcactagccagggacctggccaccttgttcagctacgccttcaacaccaaggttgtctgcatggaccagtataaggcaagcaccttggaagaggagcaactactgctggtggtgacaagcacatttgggaatggagactgtcccagcaatgggcagactctgaagaaatctctgttcatgcttagagaactcaaccacaccttcaggtatgctgtgtttggccttggctccagcatgtaccctcagttctgcgcctttgctcatgacatcgaccagaagctgtcccacctgggagcctctcagcttgccccaacaggagaaggggacgaactcagtgggcaggaggatgccttccgcagctgggctgtacaaaccttccgggcagcctgtgagacctttgatgtccgaagcaaacatcacattcagatcccgaaacgcttcacttccaatgcaacatgggagccacagcaatataggctcatccagagcccggagcctttagacctcaacagagccctcagcagcatccatgcaaagaacgtgtttaccatgaggctgaaatcccagcagaatctgcagagtgaaaagtccagccgcaccaccctcctcgttcagctcaccttcgagggcagccgagggcccagctacctgcctggggaacacctggggatcttcccaggcaaccagaccgccctggtgcagggaatcttggagcgagttgtggattgtcctacaccacaccaaactgtgtgcctggaggttctggatgagagcggcagctactgggtcaaagacaagaggctgcccccctgctcactcagccaagccctcacctacttcctggacattacgacccctcccacccagctgcagctccacaagctggctcgctttgccacggacgagacggataggcagagattggaggccttgtgtcagccctcagagtacaatgactggaagttcagcaacaaccccacgttcctggaggtgcttgaagagttcccttccttgcatgtgcccgctgccttcctgctgtcgcagctccctatcttgaagccccgctactactccatcagctcctcccaggaccacaccccctcggaggttcacctcactgtggccgtggtcacctaccgcacccgagatggtcagggtcccctgcaccatggtgtctgcagcacttggatcaggaacctgaagccccaggacccagtgccctgctttgtgcgaagtgtcagtggcttccagctccctgaggacccctcccagccttgcatcctcattgggcctggtacgggcattgctcccttccgaagtttctggcagcagcggctccatgactcccagcacaaagggctcaaaggaggccgcatgagcttggtgtttgggtgccggcacccggaggaggaccacctctatcaggaagaaatgcaggagatggtccgcaagagagtgctgttccaggtgcacacaggctactcccggctgcccggcaaacccaaggtctacgttcaggacatcctgcaaaagcagctggccaatgaggtactcagcgtgctccacggggagcagggccacctctacatttgcggagatgtgcgcatggctcgggatgtggctaccacattgaagaagctggtggccaccaagctgaacttgagcgaggagcaggtggaagactatttcttccagctcaagagccagaaacgttatcatgaagatatcttcggtgcagtcttttcctatggggcaaaaaagggcagcgccttggaggagcccaaagccacgaggctctgacagcccagagttccagcttctggcactgagtaaagataatggtgaggggcttggggagacagcgaaatgcaatcccccccaagcccctcatgtcattcccccctcctccaccctaccaagtagtattgtactattgtggactactaaatctctctcctctcctccctcccctctctccctttcctcccttcttctccactccccagctccctccttctccttctcctcctttgcctctcactcttccttggagctgagagcagagaaaaactcaacctcctgactgaagcactttgggtgaccaccaggaggcaccatgccgccgctctaatacttagctgcactatgtacagatatttatacttcatatttaagaaaacagatacttttgtctactcccaatgatggcttgggcctttcctgtataattccttgatgaaaaatatttatataaaatacattttattttaatcaaaaaaaaaaaagcggccgccaccgcggtggagctccaattcgccctatagtgagtcgtattacaattccaattgttgttgttaacttgtttattgcagcttataatggttacaaataaagcaatagcatcacaaatttcacaaataaagcatttttttcactgcattctagttgtggtttgtccaaactcatcaatgtatcttaacgcgtgatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcct